(19) United States
(12) Patent Application Publication
(10) Pub. No.: US 2014/0051085 A1
(43) Pub. Date: Feb. 20, 2014

(54) DIRECT REPROGRAMMING OF HUMAN FIBROBLASTS TO FUNCTIONAL NEURONS UNDER DEFINED CONDITIONS

(71) Applicants: Sheng Ding, Orinda, CA (US); Rajesh Ambasudhan, San Diego, CA (US)

(72) Inventors: Sheng Ding, Orinda, CA (US); Rajesh Ambasudhan, San Diego, CA (US)

(73) Assignee: The Scripps Research Institute, La Jolla, CA (US)

(21) Appl. No.: 14/001,279

(22) PCT Filed: Mar. 1, 2012
(86) PCT No.: PCT/US12/27313

§ 371 Date: Nov. 5, 2013

Related U.S. Application Data
(60) Provisional application No. 61/448,147, filed on Mar. 1, 2011.

(51) Int. Cl. C12N 5/0793; G01N 33/569; C12Q 1/68; C12N 15/85; G01N 33/487

(52) U.S. Cl. CPC C12N 5/0619; C12N 15/85; G01N 33/48728; C12Q 1/6881; G01N 33/56966
USPC 435/6.12; 435/377; 435/366; 435/455; 435/456; 435/34; 435/7.21

(57) ABSTRACT

The present invention provides methods of generating a neuronal cell from a differentiated non-neuronal cell by increasing the amount of a miR-124 microRNA, a MYT1L transcription factor, and a BRN2 transcription factor in the differentiated non-neuronal cell.

### CROSS-REFERENCES TO RELATED APPLICATIONS

The present application claims benefit of priority to U.S. Provisional Application No. 61/448,147, filed Mar. 1, 2011, which is incorporated by reference herein in its entirety.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file -47-1PC.txt, created on Feb. 21, 2012, 20,480 bytes, machine format IBM-PC, MS-Windows operating system, is hereby incorporated by reference in its entirety for all purposes.

### BACKGROUND OF THE INVENTION

The differentiated cell state is often considered stable and resistant to changes in lineage identity. However, differentiated somatic cell types from humans and other organisms have been reprogrammed to the pluripotent state (“pluripotent reprogramming”) by forced expression of a set of transcription factors (Takahashi, K. et al. Induction of pluripotent stem cells from adult human fibroblasts by defined factors. Cell 131, 861-872 (2007)), somatic cell nuclear transfer (Campbell, K. H., McWhir, J., Ritchie, W. A. & Wilmut, I. Sheep cloned by nuclear transfer from a cultured cell line. Nature 380, 64-66 (1996); Gurdon, J. B., Elsdale, T. R. & Fischberg, M. Sexually mature individuals of Xenopus laevis from the transplantation of single somatic nuclei. Nature 182, 64-65 (1958)) or cell fusion (Cowan, C. A., Atienza, J., Melton, D. A. & Eggan, K. Nuclear reprogramming of somatic cells after fusion with human embryonic stem cells. Science (New York, N.Y 309, 1369-1373 (2005); Tada, M., Takahama, Y., Abe, K., Nakatsuji, N. & Tada, T. Nuclear reprogramming of somatic cells by in vitro hybridization with ES cells. Curr Biol 11, 1553-1558 (2001)). Additionally, a few studies have demonstrated that through ectopic expression of selected genes or by cell fusion, an adult cell type can be directly converted to another adult cell type (Cobaleda, C., Jochum, W. & Busslinger, M. Conversion of mature B cells into T cells by dedifferentiation to uncommitted progenitors. Nature 449, 473-477 (2007); Davis, R. L., Weintraub, H. & Lassar, A. B. Expression of a single transfected cDNA converts fibroblasts to myoblasts. Cell 51, 987-1000 (1987); Feng, R. et al. PU.1 and C/EBPalpha/beta convert fibroblasts into macrophage-like cells. Proceedings of the National Academy of Sciences of the United States of America 105, 6057-6062 (2008); Ieda, M. et al. Direct reprogramming of fibroblasts into functional cardiomyocytes by defined factors. Cell 142, 375-386; Zhou, Q., Brown, J., Kanarek, A., Rajagopal, J. & Melton, D. A. In vivo reprogramming of adult pancreatic exocrine cells to beta-cells. Nature 455, 627-632 (2008); and Zhou, Q. & Melton, D. A. Extreme makeover: converting one cell into another. Cell stem cell 3, 382-388 (2008)). This process is termed trans-differentiation or lineage reprogramming.

Cell-replacement therapies have the potential to rapidly generate a variety of therapeutically important cell types directly from one's own easily accessible tissues, such as skin or blood. Such immunologically-matched cells would also pose less risk for rejection after transplantation. Moreover, these cells would manifest less tumorigenicity since they are terminally differentiated. However, except for one recent report on mouse cells Vierbuchen, T. et al. Direct conversion of fibroblasts to functional neurons by defined factors. Nature 463, 1035-1041 (2010)), all studies to date on lineage reprogramming have involved the conversion of one cell type to another within the same lineage (Cobaleda, C., Jochum, W. & Busslinger, M. Conversion of mature B cells into T cells by dedifferentiation to uncommitted progenitors. Nature 449, 473-477 (2007); Davis, R. L., Weintraub, H. & Lassar, A. B. Expression of a single transfected cDNA converts fibroblasts to myoblasts. Cell 51, 987-1000 (1987); Feng, R. et al. PU.1 and C/EBPalpha/beta convert fibroblasts into macrophage-like cells. Proceedings of the National Academy of Sciences of the United States of America 105, 6057-6062 (2008); Ieda, M. et al. Direct reprogramming of fibroblasts into functional cardiomyocytes by defined factors. Cell 142, 375-386; and Zhou, Q., Brown, J., Kanarek, A., Rajagopal, J. & Melton, D. A. In vivo reprogramming of adult pancreatic exocrine cells to beta-cells. Nature 455, 627-632 (2008)), a major limitation for utility in many applications.

### BRIEF SUMMARY OF THE INVENTION

In one aspect, the present invention provides methods of generating a neuronal cell from a differentiated non-neuronal cell. In some embodiments, the method comprises:
increasing the amount of a miR-124 microRNA, a MYT1L transcription factor, and a BRN2 transcription factor in the differentiated non-neuronal cell; and
culturing the differentiated non-neuronal cell in conditions suitable for neuronal differentiation; thereby generating the neuronal cell from the differentiated non-neuronal cell.

In another aspect, the present invention provides neuronal cells generated by any of the methods described herein.

In some embodiments, the differentiated non-neuronal cell is a human cell. In some embodiments, the differentiated non-neuronal cell is a somatic cell. In some embodiments, the differentiated non-neuronal cell is a fibroblast cell. In some embodiments, the differentiated non-neuronal cell is a dermal fibroblast cell. In some embodiments, the differentiated non-neuronal cell is an adult cell. In some embodiments, the differentiated non-neuronal cell is a neonatal cell.

In some embodiments, the miR-124 microRNA comprises an oligonucleotide sequence that is substantially identical to (e.g., has at least 70%, 75%, 80%, 85%, 90%, 91%, 92%, 93%, 94%, 95%, 96%, 97%, 98%, or 99% sequence identity to) any of SEQ ID NOs:1 or 4-6. In some embodiments, the miR-124 microRNA comprises SEQ ID NO:1, SEQ ID NO:4, SEQ ID NO:5, or SEQ ID NO:6.

In some embodiments, the MYT1L transcription factor comprises an amino acid sequence that is substantially identical to (e.g., has at least 70%, 75%, 80%, 85%, 90%, 91%, 92%, 93%, 94%, 95%, 96%, 97%, 98%, or 99% sequence identity to) SEQ ID NO:2. In some embodiments, the MYT1L transcription factor comprises SEQ ID NO:2.

In some embodiments, the BRN2 transcription factor comprises an amino acid sequence that is substantially identical to (e.g., has at least 70%, 75%, 80%, 85%, 90%, 91%, 92%, 93%, 94%, 95%, 96%, 97%, 98%, or 99% sequence identity to) SEQ ID NO:3. In some embodiments, the BRN2 transcription factor comprises SEQ ID NO:3.

In some embodiments, the amount of one or more of the miR-124 microRNA, MYT1L transcription factor, and BRN2 transcription factor is increased by introducing into the differentiated non-neuronal cell one or more of a first, second, and third expression cassette,
the first expression cassette comprising a promoter operably linked to a polynucleotide encoding the miR-124 microRNA;
the second expression cassette comprising a promoter operably linked to a polynucleotide encoding the MYT1L transcription factor; and
the third expression cassette comprising a promoter operably linked to a polynucleotide encoding the BRN2 transcription factor.

In some embodiments, two or more of the first, second, and third expression cassettes are introduced into the differentiated non-neuronal cell. In some embodiments, each of the first, second, and third expression cassettes is introduced into the differentiated non-neuronal cell.

In some embodiments, the promoters of the first, second, and third expression cassettes are different. In some embodiments, the promoters of at least two of the first, second, and third expression cassettes are the same promoter. In some embodiments, the promoter is an inducible promoter. In some embodiments, the expression cassette is introduced to the cell as part of a viral vector. In some embodiments, the viral vector is a lentiviral vector or an adenoviral vector.

In some embodiments, one or more of: the polynucleotide encoding miR-124, the polynucleotide encoding MYT1L, and the polynucleotide encoding BRN2 is transiently expressed in the differentiated non-neuronal cell. In some embodiments, one or more of the polynucleotide encoding miR-124, the polynucleotide encoding MYT1L, and the polynucleotide encoding BRN2 is stably expressed in the differentiated non-neuronal cell.

In some embodiments, the amount of the miR-124 microRNA is increased in the differentiated non-neuronal cell by introducing into the differentiated non-neuronal cell a polynucleotide encoding the miR-124 microRNA.

In some embodiments, the amount of one or more of the MYT1L transcription factor and BRN2 transcription factor is increased in the differentiated non-neuronal cell by introducing to the differentiated non-neuronal cell one or more of a MYT1L polypeptide and a BRN2 polypeptide.

In some embodiments, the neuronal cell is a neuron. In some embodiments, the neuron is an excitatory neuron. In some embodiments, the neuron is an inhibitory neuron.

In some embodiments, the amount of at least one of miR-124 microRNA, MYT1L transcription factor, or BRN2 transcription factor in the cell is increased for no more than 7 days. In some embodiments, the amount of at least one of miR-124 microRNA, MYT1L transcription factor, or BRN2 transcription factor in the cell is increased for no more than 4 days.

In some embodiments, the conditions that induce neuronal differentiation are chemically defined conditions. In some embodiments, the culturing step comprises contacting the differentiated non-neuronal cell with at least one of: bFGF or Noggin. In some embodiments, the culturing step further comprises contacting the differentiated non-neuronal cell with one or more of: GDNF, BDNF, and forskolin.

In some embodiments, the neuronal cell is a functional neuron. In some embodiments, the time from initiating the increase of miR-124, MYT1L, and BRN2 to the generation of the functional neuron is no more than 25 days.

In some embodiments, the neuronal cell is a mature neuron. In some embodiments, the time from initiating the increase of miR-124, MYT1L, and BRN2 to the generation of the mature neuron is no more than 20 days. In some embodiments, the time from initiating the increase of miR-124, MYT1L, and BRN2 to the generation of the mature neuron is no more than 18 days.

In some embodiments, following the culturing step the method further comprises screening the differentiated non-neuronal cell for the production of an electrical current.

In some embodiments, the method is conducted at least partly in vivo. In some embodiments, the method is conducted in vitro.

### DEFINITIONS

As used herein, the term “neuronal cell” refers to a cell of a neuronal lineage. Examples of neuronal cells include, but are not limited to, neurons, astrocytes, oligodendrocytes, and neural precursor cells.

As used herein, the term “mature neuron” refers to a differentiated neuron. In some embodiments, a neuron is said to be a mature neuron if it expresses one or more markers of mature neurons, e.g., microtubule-associated protein 2 (MAP2) and Neuronal Nuclei (NeuN).

As used herein, the term “functional neuron” refers to a differentiated neuron that is able to send or receive electrical signals. In some embodiments, a neuron is said to be a functional neuron if it exhibits electrophysiological properties (e.g., if the neuron produces excitatory postsynaptic currents, which are indicative of functional synapses, and/or produces whole-cell currents and/or neurotransmitter receptor-mediated currents) and/or if it expresses one or more markers of functional neurons, e.g., synapsin, vesicular GABA transporter (VGAT), vesicular glutamate transporter (VGLUT), and gamma-aminobutyric acid (GABA).

As used herein, a “differentiated non-neuronal cell” may refer to a cell that is not able to differentiate into all cell types of an adult organism (i.e., is not a pluripotent cell), and which is of a cellular lineage other than a neuronal lineage (e.g., a hematopoietic lineage or a connective tissue lineage). Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

As used herein, the term “potency” refers to the sum of all developmental options accessible to the cell (i.e., the developmental potency). A person of ordinary skill in the art will recognize that cell potency is a continuum, ranging from the totipotent stem cell to the terminally differentiated cell.

The continuum of cell potency includes, but is not limited to, totipotent cells, pluripotent cells, multipotent cells, oligopotent cells, and terminally differentiated cells. In the strictest sense, stem cells are either totipotent or multipotent; thus, being able to give rise to any mature cell type. However, multipotent, oligopotent, or unipotent progenitor cells are sometimes referred to as lineage-restricted stem cells (e.g., mesenchymal stem cells, adipose tissue-derived stem cells, etc.) and/or progenitor cells.

It will also be clear to one having ordinary skill in the art that potency can be partially or completely altered to any point along the developmental lineage of a cell (i.e., from totipotent to terminally differentiated cell), regardless of cell lineage.

As used herein, the term “totipotent” refers to the ability of a cell to form all cell lineages of an organism, including extraembyronic tissues. For example, in mammals the zygote is totipotent.

As used herein, the term “pluripotent” refers to the ability of a cell to form all lineages of the body or soma (i.e., the embryo proper). Thus, a pluripotent cell can differentiate into any of the three germ layers: endoderm, mesoderm, and ectoderm. For example, embryonic stem cells are a type of pluripotent cell that are able to form cells of any of the three germ layers (endoderm, mesoderm, or ectoderm).

As used herein, the term “multipotent” refers to the ability of a cell (e.g., an adult stem cell) to form multiple cell types of one lineage. For example, hematopoietic stem cells are able to form all cells of the blood cell lineage, e.g., lymphoid and myeloid cells.

As used herein, the term “oligopotent” refers to the ability of a cell (e.g., an adult stem cell) to differentiate into a few different cell types. For example, lymphoid stem cells are able to form cells of the lymphoid lineage, and myeloid stem cells are able to form cells of the myeloid lineage.

As used herein, the term “unipotent” refers to the ability of a cell to form a single cell type. For example, spermatagonial stem cells are only able to form sperm cells.

As used herein, the term “stem cell” refers to a cell that can self-renew and that has sufficient potency to differentiate into more specialized cell types. For example, an embryonic stem cell (ESC) has the capacity to self-renew indefinitely and may differentiate into any cell type of the embryo proper.

As used herein, the term “progenitor cell” refers to a cell with a limited capacity for self-renewal that spans several rounds of cell division before terminally differentiating.

As used herein, the term “self-renew” refers to the ability of a cell to go through numerous cycles of cell division while maintaining an undifferentiated state.

A “somatic cell” is a cell forming the body of an organism. Somatic cells include cells making up organs, skin, blood, bones and connective tissue in an organism, but not germ cells.

Cells can be from, e.g., human or non-human mammals. Exemplary non-human mammals include, but are not limited to, mice, rats, cats, dogs, rabbits, guinea pigs, hamsters, sheep, pigs, horses, bovines, and non-human primates. In some embodiments, a cell is from an adult human or non-human mammal. In some embodiments, a cell is from a neonatal human or non-human mammal.

As used herein, the term “direct reprogramming” or “transdifferentiation” refers to the generation of a cell of a certain lineage (e.g., a neuronal cell) from a different type of cell (e.g., a fibroblast cell) without an intermediate process of de-differentiating the cell into a cell exhibiting pluripotent stem cell characteristics.

A “microRNA” or “miRNA” refers to a non-coding nucleic acid (RNA) sequence that binds to complementary nucleic acid sequence (mRNAs) and negatively regulates the expression of the target mRNA at the post-transcriptional level. A microRNA is typically processed from a “precursor” miRNA having a double-stranded, hairpin loop structure to a “mature” form. Typically, a mature microRNA sequence is about 19-25 nucleotides in length.

A “miR-124 microRNA” refers to a precursor of miR-124 or complement thereof or a processed (i.e., mature) sequence of miR-124, or a fragment of a precursor of miR-124 comprising at least the processed sequence, or a complement thereof. In some embodiments, miR-124 microRNA comprises a processed (mature) sequence of miR-124 or a complement thereof. Mature miR-124 sequences include those sequences identified in the miRBase database, for example, hsa-miR-124 (Accession No. MIMAT0000422) (SEQ ID NO:1), mmu-miR-124 (MIMAT0000134), and rno-miR-124 (MIMAT0000828). In some embodiments, miR-124 microRNA is substantially identical (e.g, has at least 70%, 75%, 80%, 85%, 90%, 91%, 92%, 93%, 94%, 95%, 96%, 97%, 98%, or 99% sequence identity) to SEQ ID NO:1 or to any of the mature miR-124 sequences identified in the miRBase database, for example those miR-124 sequences recited herein, or a complement thereof. In some embodiments, miR-124 microRNA comprises a full-length precursor of miR-124 or a complement thereof. Full-length precursors of miR-124 include those sequences identified in the miRBase database, for example, hsa-miR-124-1 (Accession No. MI0000443) (SEQ ID NO:4), hsa-miR-124-2 (MI0000444) (SEQ ID NO:5), hsa-miR-124-3 (MI0000445) (SEQ ID NO:6), mmu-miR-124-1 (MI0000716), mmu-miR-124-2 (MI0000717), mmu-miR-124-3 (MI0000150), rno-miR-124-1 (MI0000893), rno-miR-124-2 (MI0000894), rno-miR-124-3 (MI0000892). In some embodiments, miR-124 microRNA is at least 90%, 91%, 92%, 93%, 94%, 95%, 96%, 97%, 98%, or 99% identical to any of the processed or full-length precursor miR-124 sequences identified in the miRBase database, for example those miR-124 sequences recited herein (e.g., SEQ ID NO:1, SEQ ID NO:4, SEQ ID NO:5, or SEQ ID NO:6), or a complement thereof.

A “MYT1L transcription factor” or “myelin transcription factor 1-like,” also called “Neural zinc finger 1” (“NZF1”), refers to a transcription factor having six copies of a DNA-binding zinc finger domain with a Cys-Cys-His-Cys (SEQ ID NO:7) consensus sequence, which is expressed in neurons at early stages of differentiation. The activities of MYT1 L include binding to the human myelin proteolipid protein (PLP) gene, interacting with Lingo-1 in neuronal tissue, and recruiting histone deacetylases (HDACs) to regulate neural transcription. See, e.g., Kim et al., (1997), J. Neurosci. Res. 50:272-90; Jiang et al., (1996), J. Biol. Chem. 271:10723-30; Romm et al., (2005), J. Neurochem. 93:1444-53; and Llorens et al., (2008), Dev. Neurobiol. 68:521-41. In some embodiments, a MYT1L transcription factor of the present invention comprises the amino acid sequence identified as GenBank Accession No. NP_{—}055840 (SEQ ID NO:2) or is substantially identical to (e.g., has at least 70%, 75%, 80%, 85%, 90%, 91%, 92%, 93%, 94%, 95%, 96%, 97%, 98%, or 99% sequence identity to) the MYT1L of SEQ ID NO:2. In some embodiments, variants have at least 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) to the MYT1L of SEQ ID NO:2. In some embodiments, a MYT1L transcription factor is a variant that is substantially identical to SEQ ID NO:2 and which maintains MYT1L transcription factor activity (e.g., has at least 50%, 60%, 70%, 80%, 90%, 91%, 92%, 93%, 94%, 95%, 96%, 97%, 98%, 99% or 100% activity as compared to the MYT1L of SEQ ID NO:2). In some embodiments, the MYT1L transcription factor is SEQ ID NO:2.

A “BRN2 transcription factor” or “Brain-2 transcription factor,” also called “POU domain, class 3, transcription factor 2” (“POU3F2”) or “Oct-7,” refers to a class III POU-domain transcription factor, having a DNA-binding POU domain that consists of an N-terminal POU-specific domain of about 75 amino acids and a C-terminal POU-homeo domain of about 60 amino acids, which are linked via a linker comprising a short α-helical fold, and which is predominantly expressed in the central nervous system. BRN2 is expressed in the central nervous system and interacts with the proneural basic-helix-loop-helix transcription factor Mashl to regulate aspects of neurogenesis, such as neuronal differentiation. See, e.g., Castro et al., (2006), Dev. Cell 11:831-844; Cook and Sturm, (2008), Pigment Cell Melanoma Res. 21:611-26. In some embodiments, a BRN2 transcription factor of the present invention comprises the amino acid sequence identified as GenBank Accession No. NP_{—}005595 (SEQ ID NO:3) or is substantially identical to (e.g., has at least 70%, 75%, 80%, 85%, 90%, 91%, 92%, 93%, 94%, 95%, 96%, 97%, 98%, or 99% sequence identity to) the BRN2 of SEQ ID NO:3. In some embodiments, variants have at least 90%, 91%, 92%, 93%, 94%, 95%, 96%, 97%, 98%, 99% or 100% amino acid sequence identity across the whole sequence or a portion of the sequence (e.g. a 50, 100, 150 or 200 continuous amino acid portion) to the BRN2 of SEQ ID NO:3. In some embodiments, a BRN2 transcription factor is a variant that is substantially identical to SEQ ID NO:3 and which maintains BRN2 transcription factor activity (e.g., has at least 50%, 60%, 70%, 80%, 90%, 91%, 92%, 93%, 94%, 95%, 96%, 97%, 98%, 99% or 100% activity as compared to the BRN2 of SEQ ID NO:3). In some embodiments, the BRN2 transcription factor is SEQ ID NO:3.

The term “increasing the amount of,” with respect to increasing an amount of miR-124 microRNA, MYT 1 L transcription factor, or BRN2 transcription factor, refers to increasing the quantity of the miR-124 microRNA, MYT1L transcription factor, or BRN2 transcription factor in a cell of interest (e.g., a differentiated non-neuronal cell) relative to a control. In some embodiments, the amount of miR-124, MYT1L, or BRN2 is “increased” in a cell of interest (e.g., a differentiated non-neuronal cell into which an expression cassette directing expression of a polynucleotide encoding miR-124, MYT1L, or BRN2 has been introduced) when the quantity of miR-124 microRNA, MYT1L transcription factor, or BRN2 transcription factor is at least 10%, 20%, 30%, 40%, 50%, 60%, 70%, 80%, 90%, or more relative to a control (e.g., a differentiated non-neuronal cell into which none of said expression cassettes have been introduced).

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

The term “conservatively modified variants” applies to both amino acid and nucleic acid sequences. With respect to nucleotide sequences, refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The terms “identity” or “percent identity,” in the context of two or more nucleic acids, refer to two or more sequences or subsequences that are the same or have a specified percentage of nucleotides that are the same, as measured using a BLAST or BLAST 2.0 sequence comparison algorithms with default parameters described below, or by manual alignment and visual inspection. See, e.g., the NCBI web site at ncbi.nlm.nih.gov/BLAST. Two sequences that are the same (i.e., have 100% identity) are said to be “identical.” Two sequences that have a specified percentage of nucleotides that are the same (e.g., at least about 70% identity, 75%, 80%, 85%, 90%, 91%, 92%, 93%, 94%, 95%, 96%, 97%, 98%, or 99% identity over a specified region, when compared and aligned for maximum correspondence over a comparison window or designated region) are said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The terms “protein,” “peptide,” and “polypeptide” are used interchangeably to denote an amino acid polymer or a set of two or more interacting or bound amino acid polymers.

“Expression cassette” refers to a polynucleotide comprising a promoter or other regulatory sequence operably linked to a polynucleotide sequence (e.g., a microRNA sequence or a nucleic acid sequence encoding a protein).

Expression of a transfected nucleic acid can occur transiently or stably in a cell. During “transient expression” the transfected nucleic acid is not transferred to the daughter cell during cell division. Since its expression is restricted to the transfected cell, expression of the gene is lost over time. In contrast, “stable expression” of a transfected nucleic acid can occur when the gene is co-transfected with another gene that confers a selection advantage to the transfected cell. Such a selection advantage may be a resistance towards a certain toxin that is presented to the cell.

The term “promoter,” as used herein, refers to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Promoters include constitutive and inducible promoters. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

A “vector” is a nucleic acid that is capable of transporting another nucleic acid into a cell. A vector is capable of directing expression of a protein or proteins encoded by one or more genes, or a microRNA encoded by a polynucleotide, carried by the vector when it is present in the appropriate environment.

A “viral vector” is a viral-derived nucleic acid that is capable of transporting another nucleic acid into a cell. A viral vector is capable of directing expression of a protein or proteins encoded by one or more genes, or a microRNA encoded by a polynucleotide, carried by the vector when it is present in the appropriate environment. Examples of viral vectors include, but are not limited to, retroviral, adenoviral, lentiviral and adeno-associated viral vectors.

The terms “subject” and “patient” are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but instead can refer to an individual under medical supervision.

In the context of the present invention, a “subject in need of treatment” can refer to an individual that is deficient in one or more neuronal cell populations. The deficiency can be due to a genetic defect, injury, or illness.

### BRIEF DESCRIPTION OF THE DRAWINGS

FIG. 1. Conversion of human dermal fibroblasts to human induced Neuronal (hiN) cells using defined factors under defined conditions. a, A schematic showing the experimental protocol. b, Tuj1-stained hiN cells 18 days after infection of BJ cells with the 12F pool. c, Within three days of infection, 3F (miBM)-transduced fibroblasts exhibited notable morphological changes and weak immunoreactivity with Tuj1 antibody. d, Time-lapse live images of RFP⁺ BJ cells infected with miBM showed gradual changes leading to neuronal-like morphology. e, 3F-infected BJ cells were Tuj1⁺ and exhibited characteristic neuronal morphology when stained 240 hr after infection. f-h, By day 18, hiN cells expressed in addition to Tuj1 (0, the mature neuronal markers, including MAP2 (g) and NeuN (h). i, Control cultures infected with BM transgenes along with non-specific scrambled RNA did not generate hiN cells or show immunoreactivity to Tuj1 antibody. Red: RFP; green: Tuj1 (b, c, e, f, i), MAP2 (g), or NeuN (h); blue: DAPI. Scale: 20 μm.

FIG. 2. Rapid, direct induction of hiN cells from fibroblasts without involvement of mitotic intermediates. a, Estimation of EdU positive cells in 3F (miBM)-transduced cultures that received a two hour pulse of EdU either at 2 hr or 22 hr after infection. Data are presented as mean±s.e.m. of percentage of EdU⁺ cells from 10 random fields in 3 independent experiments. ***P<0.001 (two-tailed Student's t-test). b-d, Uninfected control cultures (b), FUW rtTA (control)-infected (c), and 3F-infected cultures (d) showed comparable numbers of EdU⁺ nuclei 4 hr after infection. e-g, Dramatic reduction in EdU⁺ nuclei in 3F-infected cultures (e) compared to uninfected controls (f) or FUW rtTA-infected controls (g) 24 hr after infection, suggesting that the majority of cells were already post-mitotic by this time. h, i, After 10 days in culture under continuous presence of EdU (i.e., after 9 days of continuous exposure to EdU starting 24 hr post-infection), RFP-positive cells destined to become hiN cells incorporated virtually no EdU. Merged image of EdU staining and RFP fluorescence is shown in (h). Note the RFP⁺ cells developing neuronal morphology are negative for EdU staining j, Efficiency of hiN cell conversion from fibroblasts, estimated 18 days after infection with 3f (miBM). The percentage of hiN cells was calculated by scoring 20 randomly-selected fields (see Examples section). Data are presented as mean percent±s.e.m. of 3 independent experiments. Green: EdU⁺ nuclei (b-i); blue: Hoechst (b-g); red: RFP (b-i). Scale bar: 20 μm.

FIG. 3. Evaluation of endogenous and transgenic miR-124, BRN2, and MYT1L expression in BJ cells during the course of hiN cell generation. BJ cells were infected with lentivirus carrying inducible vectors in which transgene expression is under the control of the tetracycline operator (for Brn2 and Myth) or cumate operator (for miR-124). Both doxycycline and cumate were discontinued after 7 days. Quantitative reverse transcription (qRT)-PCR analysis was performed using cDNAs prepared from total RNA isolated from infected cells at the indicated time points. All expression levels, unless otherwise specified, were normalized to the expression levels of GAPDH expression. a, Expression levels of BRN2 and MYT1L transgenes were silenced by day 20. b, Expression of endogenous BRN2 and MYT1L at the indicated time points. c, Expression levels of virally encoded miR-124, indicating silencing of the transgene by day 20 post-infection. d, Relative expression levels of total miR-124 at various stages of hiN induction and in human neural stem/progenitor cells (hNSCs). Expression levels of miR-124 in proliferating (p) or differentiating (d) hNSCs were used as controls. e, Normalized expression levels of BAF53b, a downstream target of miR-124, indicating miR-124 activity at various stages of hiN induction and in hNSCs. BAF53b expression is limited to cells that are committed to or have already undergone neuronal differentiation. f, hiN cells displayed immunoreactivity to Tuj1 antibody when stained six days after the withdrawal of doxycycline and cumate. g, hiN cells displayed immunoreactivity to Tuj1 and MAP2 antibodies when stained 18 days after the withdrawal of doxycycline and cumate. Many of these cells also fired repetitive trains of action potentials (see FIG. 4f, right panel). Data are presented as mean±s.e.m.; experiments were performed in triplicate. Red: Tuj1; Green: MAP2; Blue: DAPI. Scale bar: 20 μm.

FIG. 4. hiN cells show functional maturation and synaptic properties. a, b, hiN cells, assessed 25 days post-infection, stained positive for synapsin-1. c, Representative traces of whole-cell currents recorded in voltage-clamp mode. Cells were hyperpolarized to −90 mV for 300 ms before applying depolarizing pulses to elicit Na⁺ and K⁺ currents. d, The inward currents could be blocked by Na⁺ channel blocker tetrodotoxin (TTX). CsCl was present in the patch electrode-filling solution to suppress K⁺ currents. Representative current traces recorded the presence of TTX are shown at left and the current-voltage (IN) relationship at right (mean±s.e.m., n=13). e, f, Representative traces of evoked (e) and spontaneous (f, left panel) action potentials recorded in current-clamp mode on day 25. In other hiN cells, repetitive trains of evoked action potentials were observed after transgene inactivation (f, right panel). g, h, Quantification of membrane capacitance (g) and membrane access resistance (h) for hiN cells (n=29). Values indicated mean±s.e.m. i-l, hiN cells expressed GABA (i, j) and its transporter VGAT (k, l). m, GABA-evoked current from hiN cell at a holding potential of −80 mV. n-p, Other hiN cells expressed the glutamate transporter VGLUT (n, o) and responded to application of exogenous NMDA at a holding potential −80 mV (p). q, r, hiN cell staining for tyrosine hydroxylase (TH) on day 25. s, Representative traces of whole-cell currents in voltage-clamp mode from aHDF-converted hiN cells 25 days post-infection. Cells were hyperpolarized to −90 mV for 300 ms before applying depolarizing pulses to elicit Na⁺ and K⁺ currents. Red: RFP; green: Synapsin (b), GABA (j), VGAT (l), VGLUT (o), TH (r). Boxed areas in the left-hand panels (a, i, k, n) are shown at higher magnification in the adjacent right-hand panels (b, j, l, o). Large red cells in i, k, n, and q are infected fibroblasts expressing RFP that have not undergone conversion to hiN cells. Scale bars: 20 μm.

FIG. 5. hiN cells derived from adult human fibroblasts (aHDF). a, A representative image of live aHDF-converted hiN cells on day 18 exhibiting typical neuronal morphology and RFP fluorescence. b-e, aHDF-converted hiN cells displayed mature neuronal markers MAP2 (b, c) and NeuN (d, e) when fixed and immunostained 18 days after 3F (miBM) infection. f, Representative traces of whole-cell currents in voltage-clamp mode from day 25 aHDF-converted hiN cells. Cells were hyperpolarized to −90 mV for 300 ms before applying depolarizing pulses to elicit Na⁺ and K⁺ currents. g, Representative action potential recorded in current-clamp mode. h, Day 25 aHDF-converted hiN cell displaying immunoreactivity for VGLUT antibody. i, Patch-clamp recording showing response of day 25 aHDF-converted hiN cell to application of exogenous NMDA. j, Spontaneous synaptic currents from aHDF hiN cell placed at high density (lower trace), reflecting mEPSCs given the composition of the intracellular and bath solutions used in the recording (see Examples). The magnified trace shows the rapid kinetics of an mEPSC. When plated at lower density to isolate the cells, aHDF hiNs were synaptically silent (upper trace). Red: RFP; green: MAP2 (c), NeuN (e) and VGLUT (h); blue: DAPI stained nuclei. b, d, and h are merged images. Scale bars: 20 μm.

FIG. 6. Absence of neural progenitor cells or neuronal cells in fibroblast populations BJ, CRL 2097, and aHDFs. a, Passage 2 fibroblasts were fixed and stained for immunoreactivity to the antigens listed. Assessments were made on the starting material (“Start,” defined as the day after initial splitting of P1 cells and culture in fibroblast medium), at one week (in D7 N4 medium), and at 18 days (representing an additional 10 days in maturation (mat.) medium). At all of these stages, BJ, CRL2097, aHDF-1, and aHDF-2 exhibited immunoreactivity to the fibroblast marker P4HA1, but no reactivity to neural progenitor cell or early neuronal markers (GFAP, SOX2, PAX6, Tuj1), peripheral/spinal neuronal markers (p75, PAX6, Nkx 2.2, Peripherin), other mature neuronal or astroglial markers (MAP2, NeuN, Synapsin, or GFAP), or an epidermal keratinocyte marker (Keratin1). Similar results were obtained when cells that had been transduced with GFP-control virus were subjected to immunostaining for the above markers. All antibodies were previously validated with appropriate positive controls. b, Reverse transcription (RT)-PCR marker analysis. Top panel, RT-PCR analysis of cDNAs prepared from total RNA isolated from Passage 2 BJ, CRL2097, aHDF cells, and human brain tissue (positive control). While SOX1 and SOX10 mRNA expression was detected in the positive control, no expression was detected in any of the fibroblast samples or in the negative controls. ACTB is a ubiquitously expressed housekeeping gene. Bottom panel, Quantitative RT-PCR analysis of cDNAs prepared from total RNA isolated after the second passage of BJ, CRL2097, aHDF-1, and aHDF-2 cells, or from positive controls (consisting of normal human whole brain tissue, normal human melanocytes, and normal human keratinocytes). While KRT1, PAX6, SOX1, SOX10, and p75 mRNA expression was detected in each of their respective positive controls, no expression was detected in any of the fibroblast samples. As expected, all the fibroblast samples manifested considerable expression levels of the fibroblast marker P4HA1. Data are presented as mean±s.e.m. of each sample in triplicate (normalized to the expression level of the ubiquitously expressed housekeeping gene GAPDH). c-e, Day 18 BJ (c), CRL2097 (d), and aHDF cells (e) infected with control GFP-vector exhibiting GFP fluorescence (green) and DAPI stained nuclei (blue). Cells were negative for Tuj1 immunoreactivity. Note the clear absence of neuronal morphology in these cells. f, Time-lapse images from live BJ cells infected with control GFP virus and subjected to the same culture conditions as 3F-infected cultures. Cells were followed from 48 hours through day 20. Note the clear absence of neuronal morphology in these cells. Scale bar: 20 μm.

FIG. 7. Effect of miR-124 alone or miR-124 combined with BRN2 or MYT1L on BJ fibroblasts. a, b, miR-124-infected fibroblasts occasionally exhibited Tuj1⁺ cells with cell bodies having multiple processes when examined on day 18. c, Tuj1⁺ cells with soma having multiple processes conspicuously increased by day 18 after infection with a combination of miR-124 and BRN2. d, Fibroblasts infected with a combination of miR-124 and MYT1L displayed Tuj1⁺ cells with an elongated morphology. None of the above combinations resulted in generation of cells with characteristic neuronal morphology or other neuronal properties. Red: RFP; green: Tuj1. Scale bar: 20 μm.

### DETAILED DESCRIPTION OF THE INVENTION

### I. Introduction

The present invention relates in part to the surprising discovery that differentiated human cells can be reprogrammed to cross lineage boundaries and directly convert to another mature and/or functional cell type. The inventors have shown herein that a differentiated human fibroblast cell can be reprogrammed into a functional neuronal cell via the expression of three factors: microRNA miR-124, transcription factor MYT1L, and transcription factor BRN2. Without being bound by a particular theory, it is believed that miR-124, a microRNA that regulates the activity of numerous anti-neuronal differentiation factors, influences neuronal cell fate determination, and when combined with specific transcription factors (MYT1L and BRN2), can facilitate lineage reprogramming to a neuronal cell.

Accordingly, the present invention provides for methods of generating a neuronal cell from a differentiated non-neuronal cell by increasing the amount of miR-124, MYT1L, and BRN2 in the differentiated non-neuronal cell and submitting the cell to defined conditions suitable for the generation of neuronal cells, e.g., culturing the cells in chemically defined medium. The present invention further provides for neuronal cells generated from differentiated non-neuronal cells according to any of the methods of the present invention.

### II. Compositions and Methods for Direct Reprogramming of Differentiated Non-Neuronal Cells to Neuronal Cells

In one aspect, the present invention provides methods for generating a neuronal cell from a differentiated non-neuronal cell. In some embodiments, the method comprises increasing the amount of a miR-124 microRNA, a MYT1L transcription factor, and a BRN2 transcription factor in the differentiated non-neuronal cell; and submitting the differentiated non-neuronal cell to conditions suitable for neuronal differentiation; thereby generating the neuronal cell from the differentiated non-neuronal cell. In some embodiments, the method is carried out with a plurality of differentiated non-neuronal cells to form a plurality (population) of neuronal cells.

In another aspect, the present invention provides neuronal cells generated from differentiated non-neuronal cells according to any of the methods described herein. In some embodiments, the neuronal cells are neurons (e.g., excitatory neurons or inhibitory neurons). In some embodiments, the neuronal cells are mature neurons. In some embodiments, the neuronal cells are functional neurons.

In particular embodiments, the invention employs routine techniques in the field of recombinant genetics. Standard recombinant methods are used for cloning, DNA and RNA isolation, amplification and purification. Generally, enzymatic reactions involving DNA ligase, DNA polymerase, restriction endonucleases and the like are performed according to the manufacturer's specifications. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

A. Increasing the Amount of miR-124, MYT1L, and BRN2

In some embodiments, the methods of the present invention comprise increasing the amount of a miR-124 microRNA, a MYT1L transcription factor, and a BRN2 transcription factor in a differentiated non-neuronal cell. The miR-124 microRNA, MYT1L transcription factor, and BRN2 transcription factor can be introduced into the differentiated non-neuronal cell, for example, by expression from a recombinant expression cassette that has been introduced into the cell. Alternatively, the MYT1L transcription factor and BRN2 transcription factor can be introduced into the differentiated non-neuronal cell by incubating the cell in the presence of exogenous MYT1L polypeptide and BRN2 polypeptide such that the polypeptides enter the cell. In still other embodiments, the miR-124 microRNA can be introduced into the differentiated non-neuronal cell by transfecting the cell with a polynucleotide encoding miR-124 or by introducing a polynucleotide encoding miR-124 into the cell via electroporation. In some embodiments, the miR-124 microRNA, MYT1L transcription factor, and BRN2 transcription factor can be introduced into the cell using a combination of methods, e.g., by introducing into the cell one or more polynucleotides encoding miR-124 microRNA, MYT1L transcription factor, and BRN2 transcription factor and one or more exogenous MYT1L and BRN2 polypeptides.

In some embodiments, the amount of miR-124, MYT1L, and BRN2 in the differentiated non-neuronal cell can be increased for a limited time, e.g., for 1, 2, 3, 4, 5, 6, 7, 8, 9, 10 or more days, or for about 2-3 weeks, for example, by expressing the miR-124, MYT1L, and BRN2 by expression cassettes under the control of an inducible promoter, then by removing the inducer after a defined period of time; or by introducing exogenous MYT1L or BRN2 polypeptide to the cell via the cell media, then changing the media after a defined period of time. In some embodiments, the amount of miR-124, MYT1L, and BRN2 in the differentiated non-neuronal cell can be increased for a prolonged or indefinite period of time, for example by stably expressing in the cell the polynucleotides encoding miR-124, MYT1L, and BRN2. In some embodiments, at least one of the miR-124 microRNA, MYT1L transcription factor, and BRN2 transcription factor is expressed in the differentiated non-neuronal cell for a shorter or longer length of time as compared to the other factors.

In some embodiments, the species of cell and nucleic acid or protein to be expressed are the same. For example, if a mouse cell is used, a mouse ortholog or variant of miR-124, MYT1L, and/or BRN2 is introduced into the cell. If a human cell is used, a human ortholog or variant of miR-124, MYT1L, and/or BRN2 is introduced into the cell. Alternatively, in some embodiments, the species of cell and nucleic acid or protein to be expressed are not the same for at least one of miR-124, MYT1L, and BRN2.

1. Polynucleotides

In some embodiments, the amount of one or more of miR-124 microRNA, MYT1L transcription factor, and BRN2 transcription factor is increased by introducing into the differentiated non-neuronal cell one or more of a first, second, and third expression cassette,
the first expression cassette comprising a promoter operably linked to a polynucleotide encoding the miR-124 microRNA;
the second expression cassette comprising a promoter operably linked to a polynucleotide encoding the MYT1L transcription factor; and
the third expression cassette comprising a promoter operably linked to a polynucleotide encoding the BRN2 transcription factor.

In some embodiments, the first expression cassette comprises a promoter operably linked to a polynucleotide encoding a miR-124 microRNA comprising a sequence that is substantially identical to (e.g., at least 70%, 75%, 80%, 85%, 90%, 91%, 92%, 93%, 94%, 95%, 96%, 97%, 98%, or 99% identical to) any of SEQ ID NOs:1 or 4-6. In particular embodiments, the polynucleotide encoding a miR-124 microRNA comprising a sequence that is at least 70%, 75%, 80%, 85%, 90%, 91%, 92%, 93%, 94%, 95%, 96%, 97%, 98%, or 99% identical to any of SEQ ID NOs:1 or 4-6 has comparable or increased activity as compared to the miR-124 microRNA activity of SEQ ID NOs:1 or 4-6. In some embodiments, the first expression cassette comprises a promoter operably linked to a polynucleotide encoding a miR-124 microRNA comprising SEQ ID NO:1, SEQ ID NO:4, SEQ ID NO:5, or SEQ ID NO:6. In some embodiments, the first expression cassette comprises a promoter operably linked to a polynucleotide encoding a miR-124 microRNA that is SEQ ID NO:1, SEQ ID NO:4, SEQ ID NO:5, or SEQ ID NO:6.

In some embodiments, the second expression cassette comprises a promoter operably linked to a polynucleotide encoding a MYT1L transcription factor comprising a sequence that is substantially identical to (e.g., at least 70%, 75%, 80%, 85%, 90%, 91%, 92%, 93%, 94%, 95%, 96%, 97%, 98%, or 99% identical to) SEQ ID NO:2. In particular embodiments, the polynucleotide encoding a MYT1L transcription factor comprising a sequence that is at least 70%, 75%, 80%, 85%, 90%, 91%, 92%, 93%, 94%, 95%, 96%, 97%, 98%, or 99% identical to SEQ ID NO:2 has comparable or increased activity as compared to the activity of a MYT1L polypeptide as set forth in SEQ ID NO:2. In some embodiments, the second expression cassette comprises a promoter operably linked to a polynucleotide encoding a MYT1L transcription factor comprising SEQ ID NO:2. In some embodiments, the second expression cassette comprises a promoter operably linked to a polynucleotide encoding a MYT1L transcription factor that is SEQ ID NO:2.

In some embodiments, the third expression cassette comprises a promoter operably linked to a polynucleotide encoding a BRN2 transcription factor comprising a sequence that is substantially identical to (e.g., at least 70%, 75%, 80%, 85%, 90%, 91%, 92%, 93%, 94%, 95%, 96%, 97%, 98%, or 99% identical to) SEQ ID NO:3. In particular embodiments, the polynucleotide encoding a BRN2 transcription factor comprising a sequence that is at least 70%, 75%, 80%, 85%, 90%, 91%, 92%, 93%, 94%, 95%, 96%, 97%, 98%, or 99% identical to SEQ ID NO:3 has comparable or increased activity as compared to the activity of a BRN2 polypeptide as set forth in SEQ ID NO:3. In some embodiments, the third expression cassette comprises a promoter operably linked to a polynucleotide encoding a BRN2 transcription factor comprising SEQ ID NO:3. In some embodiments, the third expression cassette comprises a promoter operably linked to a polynucleotide encoding a BRN2 transcription factor that is SEQ ID NO:3.

In some embodiments, two or more of the first, second, and third expression cassettes are introduced into the differentiated non-neuronal cell. In some embodiments, each of the first, second, and third expression cassettes is introduced into the differentiated non-neuronal cell.

It will be appreciated that as an alternative to expressing each of miR-124, MYT1L, and BRN2 in a separate expression cassette, an expression cassette can be used that expresses more than one polynucleotide. For example, where one expression cassette expresses multiple polynucleotides (i.e., two or more of a polynucleotide encoding the miR-124 microRNA, a polynucleotide encoding the MYT1L transcription factor, and a polynucleotide encoding the BRN2 transcription factor), a polycistronic expression cassette can be used.

In one embodiment, a polycistronic expression cassette comprises a polynucleotide encoding each of miR-124 microRNA, MYT1L, and BRN2 in any order. The portions of the polynucleotide sequence encoding miR-124 microRNA, MYTL1L, and BRN2 may be separated by internal ribosome entry sites (IRES) and/or polynucleotide sequences encoding self-cleaving viral polypeptides, e.g., 2A peptides from the foot and mouth disease virus (FMDV), in any combination. For example, a polycistronic expression cassette may comprise an IRES sequence separating miR-124 microRNA from MYT1L and BRN2 and a self-cleaving viral polypeptide separating the polynucleotide sequences encoding MYT1L and BRN2. In another embodiment, a polycistronic expression cassette comprises a polynucleotide sequence encoding MYT1L and BRN2, separated by an IRES or self-cleaving viral polypeptide, and a 3′ UTR comprising the polynucleotide sequence encoding the miR-124 microRNA.

Where multiple expression cassettes are used, the same promoter can be used for each of the expression cassettes, or at least for two of the expression cassettes, or alternatively a different promoter can be used for each of the expression cassettes. In some embodiments, the promoter is an inducible promoter. Examples of inducible promoters are known in the art and include, but are not limited to, tetracycline- or doxycycline-inducible promoters. In some embodiments, the inducible promoter induces expression of miR-124, MYT1L, and/or BRN2 in the presence of doxycycline via a tetracycline inducible operator tetO in conjunction with a reverse tet transactivator.

Any type of vector can be used to introduce an expression cassette of the invention into a cell. Exemplary vectors include but are not limited to plasmids, piggyBAC transposons, and viral vectors. Exemplary viral vectors include, e.g., adenoviral vectors, AAV vectors, and retroviral (e.g., lentiviral) vectors.

Suitable methods for nucleic acid delivery for transformation of a cell, a tissue or an organism for use with the current invention are believed to include virtually any method by which a nucleic acid (e.g., DNA) can be introduced into a cell, a tissue or an organism, as described herein or as would be known to one of ordinary skill in the art (e.g., Stadtfeld and Hochedlinger, Nature Methods 6(5):329-330 (2009); Yusa et al., Nat. Methods 6:363-369 (2009); Woltjen, et al., Nature 458, 766-770 (9 Apr. 2009)). Such methods include, but are not limited to, direct delivery of DNA such as by ex vivo transfection (Wilson et al., Science, 244:1344-1346, 1989, Nabel and Baltimore, Nature 326:711-713, 1987), optionally with a lipid-based transfection reagent such as Fugene6 (Roche) or Lipofectamine (Invitrogen), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van Der Eb, Virology, 52:456-467, 1973; Chen and Okayama, Mol. Cell Biol., 7(8):2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., J Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); and any combination of such methods, each of which is incorporated herein by reference.

To address the safety issues that arise from target cell genomes harboring integrated exogenous sequences, a number of modified genetic protocols have been further developed and can be modified according to the present invention to generate neuronal cells with potentially reduced risks. These protocols include, for example, non-integrating adenoviruses to deliver reprogramming genes (Stadtfeld, M., et al. (2008) Science 322, 945-949), transient transfection of reprogramming plasmids (Okita, K., et al. (2008) Science 322, 949-953), piggyBac transposition systems (Woltjen, K., et al. (2009). Nature 458, 766-770, Yusa et al. (2009) Nat. Methods 6:363-369, Kaji, K., et al. (2009) Nature 458, 771-775), Cre-excisable viruses (Soldner, F., et al. (2009) Cell 136, 964-977), and oriP/EBNA1-based episomal expression system (Yu, J., et al. (2009) Science DOI: 10.1126).

In some embodiments, miR-124 microRNA is introduced to a differentiated non-neuronal cell by transfecting the cell with a polynucleotide encoding miR-124 comprising a sequence that is substantially identical to (e.g., at least 70%, 75%, 80%, 85%, 90%, 91%, 92%, 93%, 94%, 95%, 96%, 97%, 98%, or 99% identical to) any of SEQ ID NOs:1 or 4-6. In some embodiments, miR-124 microRNA is introduced to a differentiated non-neuronal cell by transfecting the cell with a polynucleotide encoding miR-124 comprising SEQ ID NO:1, SEQ ID NO:4, SEQ ID NO:5, or SEQ ID NO:6.

In some embodiments, miR-124 microRNA is introduced to a differentiated non-neuronal cell by electroporating the cell with a polynucleotide encoding miR-124 comprising a sequence that is substantially identical to (e.g., at least 70%, 75%, 80%, 85%, 90%, 91%, 92%, 93%, 94%, 95%, 96%, 97%, 98%, or 99% identical to) any of SEQ ID NOs:1 or 4-6. In some embodiments, miR-124 microRNA is introduced to a differentiated non-neuronal cell by electroporating the cell with a polynucleotide encoding miR-124 comprising SEQ ID NO:1, SEQ ID NO:4, SEQ ID NO:5, or SEQ ID NO:6.

2. Exogenous Proteins

In some embodiments, the amount of one or more of the MYT1L transcription factor and BRN2 transcription factor is increased by introducing into the differentiated non-neuronal cell one or more of a MYT1L polypeptide and a BRN2 polypeptide.

In some embodiments, the MYT1L polypeptide comprises a sequence that is substantially identical to (e.g., at least 70%, 75%, 80%, 85%, 90%, 91%, 92%, 93%, 94%, 95%, 96%, 97%, 98%, or 99% identical to) SEQ ID NO:2, wherein the MYT1L polypeptide has comparable or increased activity as compared to the activity of a MYT1L polypeptide as set forth in SEQ ID NO:2. In some embodiments, the MYT1L polypeptide comprises SEQ ID NO:2. In some embodiments, the MYT1L polypeptide is SEQ ID NO:2.

In some embodiments, the BRN2 polypeptide comprises a sequence that is substantially identical to (e.g., at least 70%, 75%, 80%, 85%, 90%, 91%, 92%, 93%, 94%, 95%, 96%, 97%, 98%, or 99% identical to) SEQ ID NO:3, wherein the BRN2 polypeptide has comparable or increased activity as compared to the activity of a BRN2 polypeptide as set forth in SEQ ID NO:3. In some embodiments, the BRN2 polypeptide comprises SEQ ID NO:3. In some embodiments, the BRN2 polypeptide is SEQ ID NO:3.

In some embodiments, the MYT1L and/or BRN2 polypeptide is linked (e.g., linked as a fusion protein or otherwise covalently or non-covalently linked) to a polypeptide that enhances the ability of the transcription factor to enter the cell (and in some embodiments the cell nucleus). For example, in some embodiments, the MYT1L comprises a polypeptide sequence of SEQ ID NO:2 or substantially identical to SEQ ID NO:2 linked to a polypeptide that enhances the ability of MYT1L to enter the cell. In some embodiments, the BRN2 comprises a polypeptide sequence of SEQ ID NO:3 or substantially identical to SEQ ID NO:3 linked to a polypeptide that enhances the ability of BRN2 to enter the cell.

A number of polypeptides capable of mediating introduction of associated molecules into a cell have been described previously and can be adapted to the present invention. See, e.g., Langel (2002) Cell Penetrating Peptides: Processes and Applications, CRC Press, Pharmacology and Toxicology Series. Examples of polypeptide sequences that enhance transport across membranes include, but are not limited to, the Drosophila homeoprotein antennapedia transcription protein (AntHD) (Joliot et al., New Biol. 3: 1121-34, 1991; Joliot et al., Proc. Natl. Acad. Sci. USA, 88: 1864-8, 1991; Le Roux et al., Proc. Natl. Acad. Sci. USA, 90: 9120-4, 1993), the herpes simplex virus structural protein VP22 (Elliott and O'Hare, Cell 88: 223-33, 1997); the HIV-1 transcriptional activator TAT protein (Green and Loewenstein, Cell 55: 1179-1188, 1988; Frankel and Pabo, Cell 55: 1 289-1193, 1988); Kaposi FGF signal sequence (kFGF); protein transduction domain-4 (PTD4); Penetratin, M918, Transportan-10; a nuclear localization sequence, a PEP-I peptide; an amphipathic peptide (e.g., an MPG peptide); delivery enhancing transporters such as described in U.S. Pat. No. 6,730,293 (including but not limited to an peptide sequence comprising at least 5-25 or more contiguous arginines or 5-25 or more arginines in a contiguous set of 30, 40, or 50 amino acids; including but not limited to an peptide having sufficient, e.g., at least 5, guanidino or amidino moieties); and commercially available Penetratin™ 1 peptide, and the Diatos Peptide Vectors (“DPVs”) of the Vectocell® platform available from Daitos S.A. of Paris, France. See also, WO/2005/084158 and WO/2007/123667 and additional transporters described therein. Not only can these proteins pass through the plasma membrane but the attachment of other proteins, such as the transcription factors described herein, is sufficient to stimulate the cellular uptake of these complexes.

The MYT1L and/or BRN2 polypeptides can be introduced to the cell by traditional methods such as lipofection, electroporation, calcium phosphate precipitation, particle bombardment and/or microinjection, or can be introduced into cells by a protein delivery agent. For example, the exogenous polypeptide can be introduced into cells by covalently or noncovalently attached lipids, e.g., by a covalently attached myristoyl group. Lipids used for lipofection are optionally excluded from cellular delivery modules in some embodiments. In some embodiments, the transcription factor polypeptides described herein are exogenously introduced as part of a liposome, or lipid cocktail (such as commercially available Fugene6 and Lipofectamine). In another alternative, the transcription factor proteins can be microinjected or otherwise directly introduced into the target cell. In some embodiments, the transcription factor polypeptides are delivered into cells using Profect protein delivery reagents, e.g., Profect-P1 and Profect-P2 (Targeting Systems, El Cajon, Calif.), or using Pro-Ject transfection reagents (Pierce, Rockford Ill., USA). In some embodiments, the transcription factor polypeptides are delivered into cells using a single-wall nano tube (SWNT).

In some embodiments, a combination of methods can be used to introduce miR-124 microRNA, MYT1L transcription factor, and BRN2 transcription factor into the differentiated non-neuronal cell. For example, in some embodiments, a polynucleotide encoding miR-124 microRNA; a MYT1L polypeptide; and a BRN2 polypeptide are introduced into the cell. In some embodiments, an expression cassette comprising a promoter operably linked to a polynucleotide encoding miR-124 microRNA; a MYT1L polypeptide; and a BRN2 polypeptide are introduced into the cell. In some embodiments, a first expression cassette comprising a promoter operably linked to a polynucleotide encoding miR-124 microRNA; a second expression cassette comprising a promoter operably linked to a polynucleotide encoding one of MYT1L transcription factor or BRN2 transcription factor; and a polypeptide comprising the other of MYT1L transcription factor or BRN2 transcription factor are introduced into the cell.

B. Methods of Obtaining Differentiated Non-Neuronal Cells

Differentiated non-neuronal cells for direct reprogramming into neuronal cells can be obtained from any mammal, e.g., a rodent, rabbit, goat, bovine, sheep, horse, non-human primate or human. In some embodiments, the differentiated non-neuronal cells are obtained from a human. In some embodiments, the differentiated non-neuronal cells are obtained from an adult mammal (e.g., human). In some embodiments, the differentiated non-neuronal cells are obtained from a neonatal mammal (e.g., human).

Differentiated non-neuronal cells can be derived from any of a number of tissues, e.g., connective tissue, adipose tissue, epithelial tissue, etc. In some embodiments, the differentiated non-neuronal cells are derived from connective tissue. In some embodiments, the differentiated non-neuronal cells are fibroblasts, e.g., dermal fibroblasts, derived for example from the dermis of neonatal foreskin or adult skin. Methods of isolating and culturing dermal fibroblasts are generally known in the art; see, e.g., Mansbridge, “Dermal Fibroblasts,” in Human Cell Culture vol. 5 (2002), pages 125-172.

For therapeutic applications of the neuronal cells that are generated by direct reprogramming, the differentiated non-neuronal cells can be obtained from the intended recipient of the neuronal cell transplant. That is, the differentiated non-neuronal cells and neuronal cells will be autologous to the recipient of the neuronal cells (i.e., derived from or originating in the recipient). In some aspects, the differentiated non-neuronal cells can instead be obtained from a different individual or group of individuals, e.g., a close relative. In that case, the differentiated non-neuronal cells and neuronal cells will be allogeneic to the recipient of the neuronal cells (i.e., derived from, originating in, or being members of the same species, where the members are genetically related or genetically unrelated but genetically similar).

C. Methods of Culturing Differentiated Non-Neuronal Cells for Direct Reprogramming

In particular embodiments, differentiated non-neuronal cells into which a miR-124 microRNA, a MYT1L transcription factor, and a BRN2 transcription factor have been introduced can be cultured according to standard cell culture conditions suitable for neuronal differentiation. In particular embodiments, submitting a differentiated non-neuronal cell to conditions suitable for neuronal differentiation includes, but is not limited to, culturing the non-neuronal cells according to cell culture conditions suitable for neuronal differentiation, as described herein, or as otherwise known in the art. Examples of suitable cell culture conditions are described in the Examples section. Other exemplary cell culture conditions are described in more detail, e.g., in Picot, Human Cell Culture Protocols (Methods in Molecular Medicine) 2010 ed., and Davis, Basic Cell Culture 2002 ed.

In some embodiments, the cells are cultured in induction media, comprising at least one of basic fibroblast growth factor (bFGF) or Noggin, for about 4-7 days, during which time the miR-124, MYT1L, and BRN2 are introduced into the cell (e.g., using one or more expression cassettes to control expression of the miR-124, MYT1L, and BRN2, or by introducing MTY1L and/or BRN2 exogenous polypeptides to the cell; or by introducing a polynucleotide encoding miR-124 into the cell; or using a combination of any of these methods). In some embodiments, induction media comprises a normal growth medium (e.g., DMEM/F12 supplemented with N2 and/or B27) and bFGF and/or Noggin, wherein the amount of bFGF and/or Noggin in the medium is from about 10 ng/ml to about 200 ng/ml (e.g., about 10, 20, 30, 40, 50, 60, 70, 80, 90, 100, 110, 120, 130, 140, 150, 160, 170, 180, 190, or 200 ng/ml). In some embodiments, induction media comprises a normal growth medium (e.g., DMEM/F12 supplemented with N2 and/or B27) and bFGF and Noggin.

In some embodiments, following culturing in induction media the cells are subsequently cultured in neuronal differentiation media comprising one or more of glial cell-derived neurotrophic factor (GDNF), brain-derived neurotrophic factor (BDNF), and forskolin. In some embodiments, one or more of miR-124, MYT1L, and BRN2 are expressed in the cell for at least a portion of the time in which the cells are cultured in neuronal differentiation media (e.g., using one or more expression cassettes to control expression of the miR-124, MYT1L, and BRN2, or by introducing MTY1L and/or BRN2 exogenous polypeptides to the cell; or by introducing a polynucleotide encoding miR-124 into the cell; or using a combination of any of these methods). In some embodiments, one or more of miR-124, MYT1L, and BRN2 are not introduced into the cell during the time in which the cells are cultured in neuronal differentiation media. In some embodiments, neuronal differentiation media comprises a normal growth medium (e.g., DMEM/F12 supplemented with N2 and/or B27) and one or more of GDNF, BDNF, and/or forskolin, wherein the amount of GDNF and/or BDNF in the medium is from about 10 ng/ml to about 200 ng/ml (e.g., about 10, 20, 30, 40, 50, 60, 70, 80, 90, 100, 110, 120, 130, 140, 150, 160, 170, 180, 190, or 200 ng/ml) and the amount of forskolin in the medium is from about 0.001 mg/ml to about 10 mg/ml (e.g., about 0.001, 0.005, 0.01, 0.05, 0.1, 0.5, 1, 2, 3, 4, 5, 6, 7, 8, 9, or 10 mg/ml). In some embodiments, neuronal differentiation media comprises a normal growth medium (e.g., DMEM/F12 supplemented with N2 and/or B27) and GDNF, BDNF, and forskolin.

In some embodiments, the cells are cultured in induction media with one or more of miR-124, MYT1L, and BRN2 for no more than 4 days. In some embodiments, the cells are cultured in induction media with one or more of miR-124, MYT1L, and BRN2 for no more than 5 days. In some embodiments, the cells are cultured in induction media with one or more of miR-124, MYT1L, and BRN2 for no more than 6 days. In some embodiments, the cells are cultured in induction media with one or more of miR-124, MYT1L, and BRN2 for no more than 7 days.

In some embodiments, the cells are cultured in neuronal differentiation media for about 10-20 days. In some embodiments, the cells are cultured in neuronal differentiation media for about 10-14 days.

In some embodiments, the length of time from initiating the increase of miR-124, MYT1L, and BRN2 to the generation of the neuronal cell is no more than 25 days. In some embodiments, the neuronal cell that is generated is a functional neuron.

In some embodiments, the length of time from initiating the increase of miR-124, MYT1L, and BRN2 to the generation of the neuronal cell is no more than 20 days. In some embodiments, the length of time from initiating the increase of miR-124, MYT1L, and BRN2 to the generation of the neuronal cell is no more than 18 days. In some embodiments, the neuronal cell that is generated is a mature neuron.

D. Methods of Screening for Mature Neurons and Functional Neurons

In some embodiments, the method further comprises, after the step of submitting the differentiated non-neuronal cell to conditions suitable for neuronal differentiation (e.g., culturing the differentiated non-neuronal cell in conditions suitable for neuronal differentiation), screening the cell for the presence of a mature neuron and/or a functional neuron.

Mature neurons can be identified by detecting the presence of one or more biological markers. In some embodiments, a mature neuron is identified by detecting the presence or level of expression of one or more of microtubule-associated protein 2 (MAP2) or Neuronal Nuclei (NeuN).

Functional neurons can be identified by detecting the presence of one or more biological markers and/or by measuring for the production of electrical currents. In some embodiments, a functional neuron is identified by detecting the presence or level of expression of one or more of synapsin, vesicular GABA transporter (VGAT), vesicular glutamate transporter (VGLUT), or gamma-aminobutyric acid (GABA). In some embodiments, a functional neuron is identified by the production of one or more of an excitatory postsynaptic current, an action potential, a whole-cell current, or a neurotransmitter receptor-mediated current.

Methods for detecting the level of expression of a marker can be carried out, for example, using standard nucleic acid or protein detection techniques known in the art. Detection can be accomplished by labeling a nucleic acid probe or a primary antibody or secondary antibody with, for example, a radioactive isotope, a fluorescent label, an enzyme or any other detectable label known in the art.

For example, immunoassays, such as enzyme-linked immunosorbent assay (ELISA), immunofluorescence (IF), and chemiluminescence assays (CL) can be used to detect the level of expression of a protein marker in a sample of interest. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000.

Analysis of nucleic acid markers can also be performed using techniques known in the art including, without limitation, microarrays, polymerase chain reaction (PCR)-based analysis, sequence analysis, and electrophoretic analysis. In some embodiments, RT-PCR is used according to standard methods known in the art. In other embodiments, qPCR and nucleic acid microarrays can be used to detect nucleic acids. Reagents that bind to selected markers of interest can be prepared according to methods known to those of skill in the art or purchased commercially.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

For detecting the production of an electrical current, standard electrophysiological methods known in the art can be used. For example, patch clamp recordings can be recorded on a cell of interest using voltage-clamp mode (to record changes in current) or in current-clamp mode (to record changes in membrane voltage). Suitable conditions for patch clamp recordings are described in the Examples below. Patch clamp techniques are also described generally in Walz et al., Patch-Clamp Analysis: Advanced Techniques, Humana Press Inc., 2002; and Molnar and Hickman, Patch-Clamp Methods and Protocols, Humana Press Inc., 2007.

### III. Methods of Treatment

In another aspect, the present invention provides methods of treating a subject in need thereof with a neuronal cell or population of neuronal cells generated by any of the methods described herein. In some embodiments, the method comprises transplanting the neuronal cell or cells into the subject. In some embodiments, the subject in need thereof has a neurodegenerative disease. A “neurodegenerative disease or condition,” as used herein, is a disease or medical condition associated with neuron loss or dysfunction. Examples of neurodegenerative diseases or conditions include neurodegenerative diseases, brain injuries, spinal cord injuries, or CNS dysfunctions. Neurodegenerative diseases include, for example, demyelination diseases, Alzheimer's disease, age-related dementia, multiple sclerosis (MS), macular degeneration, glaucoma, diabetic retinopathy, peripheral neuropathy, Huntington's disease, amyotrophic lateral sclerosis (ALS), and Parkinson's disease. Brain injuries include, for example, stroke (e.g., hemorrhagic stroke, focal ischemic stroke or global ischemic stroke) and traumatic brain injuries (e.g. injuries caused by a brain surgery or physical accidents). Spinal cord injuries include traumatic injuries caused by surgery or physical accidents. CNS dysfunctions include, for example, major depression, bipolar disorder, epilepsy, anxiety, neurosis, and psychotic disorders such as schizophrenia.

In some embodiments, the method of treatment comprises:
increasing the amount of a miR-124 microRNA, a MYT1L transcription factor, and a BRN2 transcription factor in the differentiated non-neuronal cell;
submitting the differentiated non-neuronal cell to conditions suitable for neuronal differentiation; thereby generating the neuronal cell from the differentiated non-neuronal cell; and
administering the neuronal cell to a subject in need thereof.

In some embodiments, the neuronal cell is a neuron. In some embodiments, the neuron is a mature neuron. In some embodiments, the neuron is a functional neuron.

In some embodiments, the differentiated non-neuronal cell is a somatic cell. In some embodiments, the differentiated non-neuronal cell is a fibroblast cell. In some embodiments, the differentiated non-neuronal cell is a dermal fibroblast cell.

In some embodiments, the differentiated non-neuronal cell is from the subject in need thereof (i.e., is autologous to the subject in need thereof). In some embodiments, the differentiated non-neuronal cell is not from the subject in need thereof (i.e., is allogenic to the subject in need thereof).

In particular embodiments, the neuronal cells to be administered to the subject in need thereof can be administered according to any known method in the art. In some embodiments, the neuronal cell or cells are administered by oral administration, administration as a suppository, topical contact, parenteral, intravenous, intraperitoneal, intramuscular, intralesional, intranasal or subcutaneous administration, intrathecal administration, or the implantation of a slow-release device, e.g., a mini-osmotic pump, to the subject.

In some embodiments, the neuronal cells are administered to the subject by injection, e.g., intravenously. The administration can be either in a bolus or in an infusion. The neuronal cell compositions of the invention can comprise a pharmaceutically acceptable carrier. Pharmaceutically acceptable carriers are determined in part by the particular method used to administer the composition, but are typically isotonic, buffered saline solutions. Accordingly, there are a wide variety of suitable formulations of pharmaceutical compositions of the present invention (see, e.g., Remington: The Science and Practice of Pharmacy, 21st Edition, Baltimore, Md.: Lippincott Williams & Wilkins, 2006). The neuronal cell compositions of the invention can be administered in a single dose, multiple doses, or on a regular basis (e.g., daily) for a period of time (e.g., 2, 3, 4, 5, 6, days, weeks, months, or as long as the condition persists).

The dose administered to the subject, in the context of the present invention should be sufficient to effect a beneficial response in the subject over time, e.g., a reduction of neurodegenerative symptoms. The optimal dose level for any patient will depend on a variety of factors including the efficacy of the specific composition employed, the age, body weight, physical activity, and diet of the patient, on a possible combination with other drugs, and on the severity of the neurodegenerative disorder or condition. The size of the dose also will be determined by the existence, nature, and extent of any adverse side-effects that accompany the administration of the neuronal cells in a particular subject.

In some embodiments, the method of treatment further comprises obtaining differentiated non-neuronal cells from the subject prior to treatment and directly reprogramming the differentiated non-neuronal cells into neuronal cells according to any of the methods described herein. In some aspects, differentiated non-neuronal cells are harvested more than once, or routinely, and freshly reprogrammed directly into neuronal cells prior to administration (reintroduction) into the subject.

Aqueous solutions of the neuronal cells can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, e.g., sodium acetate, sodium lactate, sodium chloride, potassium chloride, calcium chloride, sorbitan monolaurate, and triethanolamine oleate. Sugars can also be included for stabilizing the compositions, such as a stabilizer for lyophilized compositions. In some embodiments, the neuronal cells can be preserved at −20 C or −70 C in a standard preservation solution comprising, e.g., DMSO.

### IV. Kits

In another aspect, the present invention provides kits for direct reprogramming of differentiated non-neuronal cells into neuronal cells. The kit can optionally include written instructions or electronic instructions (e.g., on a CD-ROM or DVD). In some aspects, kits of the invention will include a case or container for holding the reagents in the kit, which can be included separately or in combination.

In some embodiments, the kit includes reagents for isolating differentiated non-neuronal cells (e.g., from a primary tissue from a human or non-human mammal); reagents for direct reprogramming (e.g., a polynucleotide sequence encoding miR-124, a polynucleotide sequence encoding MYT1L, and/or a polynucleotide sequence encoding BRN2, optionally in one or more expression cassettes; MYT1L and/or BRN2 polypeptides; etc.); transfection reagents; and/or reagents for culturing the cells under conditions suitable for neuronal differentiation (e.g., cell culture media, media supplements, recombinant proteins for promoting neuronal differentiation as described herein, tissue culture plates or bottles, etc.). In some embodiments, the kit further comprises nucleic acid or antibody probes for detecting the presence of a mature neuron and/or a functional neuron. The kit can optionally include a device for collecting the subject sample. The kit can also include tubes or other containers for holding the sample during processing.

### EXAMPLES

The following examples are offered to illustrate, but not to limit the claimed invention.

### Example 1

In this study human primary dermal fibroblasts were directly reprogrammed to functional neurons using specific factors under defined conditions.

### A Twelve-Factor Pool Reprogrammed Human Fibroblasts to Neurons

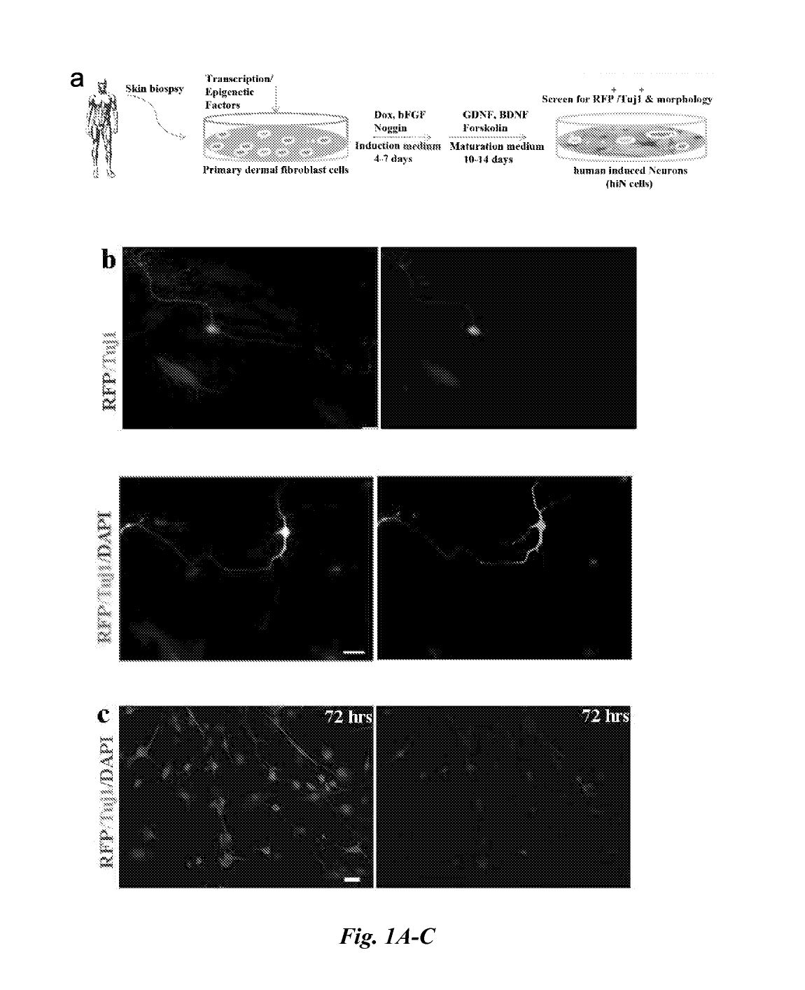

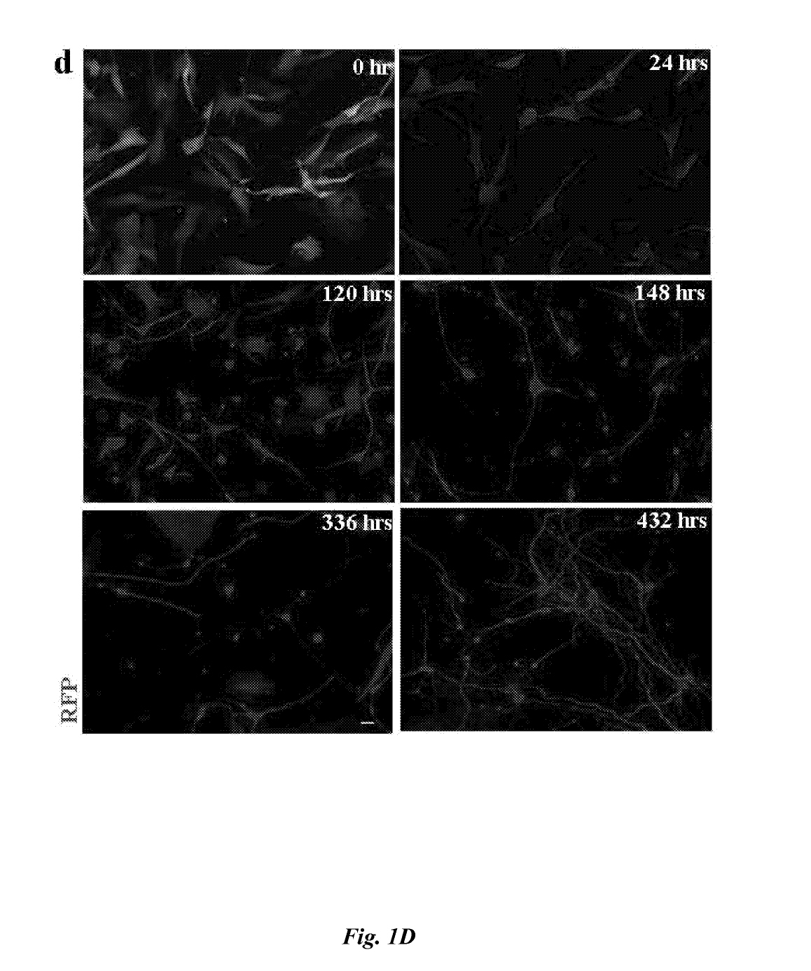

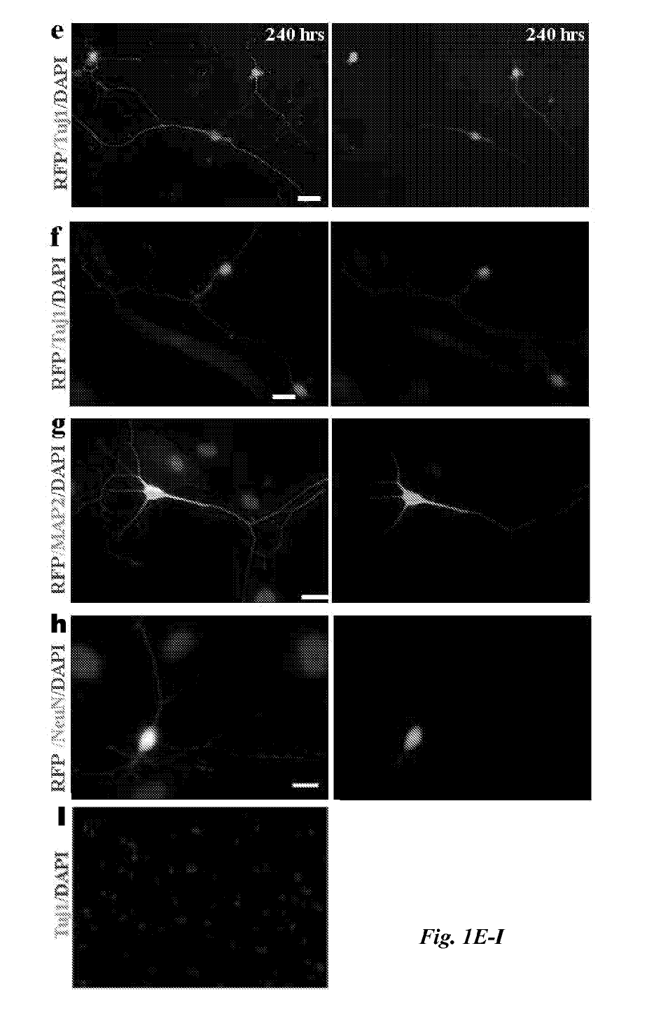

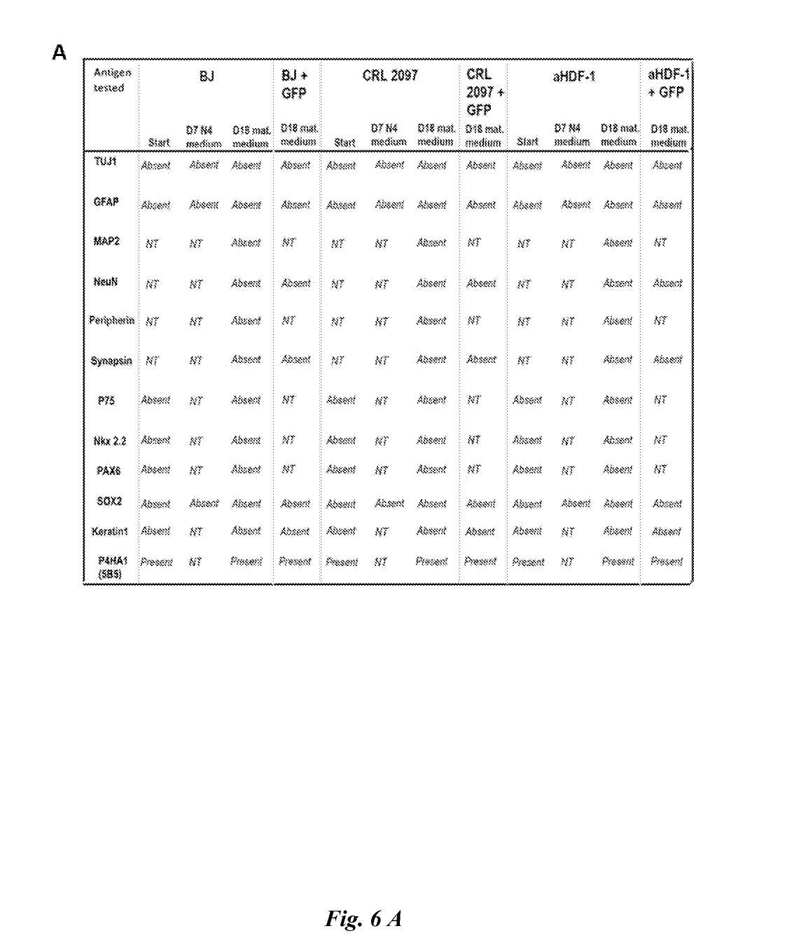

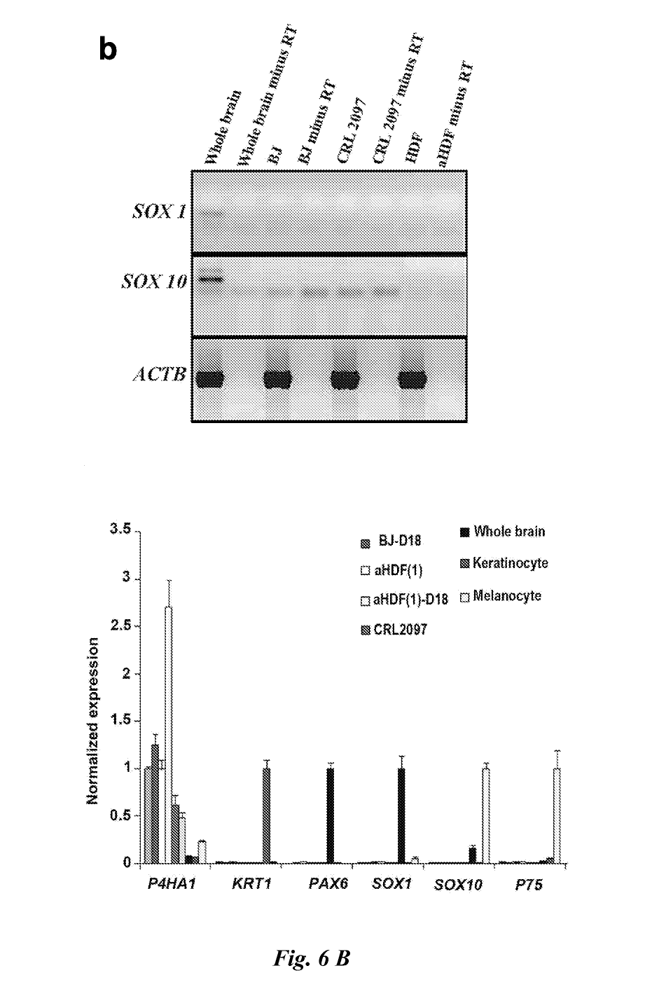

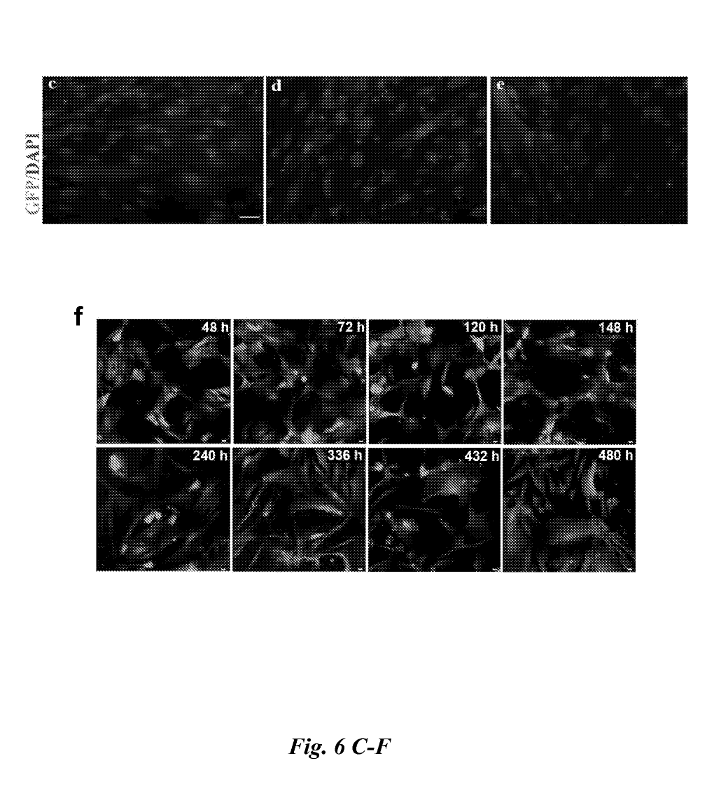

Given the roles of specific transcription factors, signaling molecules, and microRNAs in neuronal lineage determination during development or cell fate regulation, eleven transcription factors (Table 1) and a microRNA (miR-124) were picked to test for their ability to convert primary human fibroblasts, BJ or CRL2097, to functional neurons. The absence of any contaminating neural or neuronal cells in BJ and CRL2097 cell cultures was confirmed by immunostaining (FIG. 6a) and RT-PCR (FIG. 6b). The fibroblast cells were transduced with lentiviruses carrying the twelve factors pooled together (12F pool, with equal representation of each). The details of subsequent culture conditions are depicted in the schematic diagram in FIG. 1a and in the Methods section. Red fluorescent protein (RFP) co-expressed from the miRNA vector (pLemiR) was used to monitor morphological changes that occurred in the cells that were successfully infected. Eighteen days after viral transduction (seven days in induction medium and a further eleven days under differentiation conditions), the cells were fixed and immunostained for the early neuronal marker βIII-tubulin. Tuj1 immunoreactivity was not observed in uninfected (FIG. 6a) or GFP-control cells (FIG. 6c-f), but a few Tuj1/RFP double-positive cells manifesting a typical neuronal morphology could be observed in the 12F infected cultures (FIG. 1b). These findings suggested that factors in the 12F pool had the capacity to directly reprogram human fibroblasts to cells manifesting a typical neuronal morphology.

TABLE 1
List of transcription factors that were tested for reprogramming

| | |
|---|---|
| ASCL1 | NM_004316.3 |
| BRG1 | NM_001128844.1 |
| BMI1 | NM_005180.6 |
| BRN2 | NM_005604.2 |
| EMX2 | NM_001165924.1 |
| MYT1L | NM_015025.2 |
| HES6 | NM_001142853.1 |
| PRKCI | NM_002740.5 |
| PAX6 | NM_000280.3 |
| SOX1 | NM_005986.2 |
| SOX2 | NM_003106 |

At Least Three Factors were Required for Human Induced Neuronal (hiN) Cell Generation

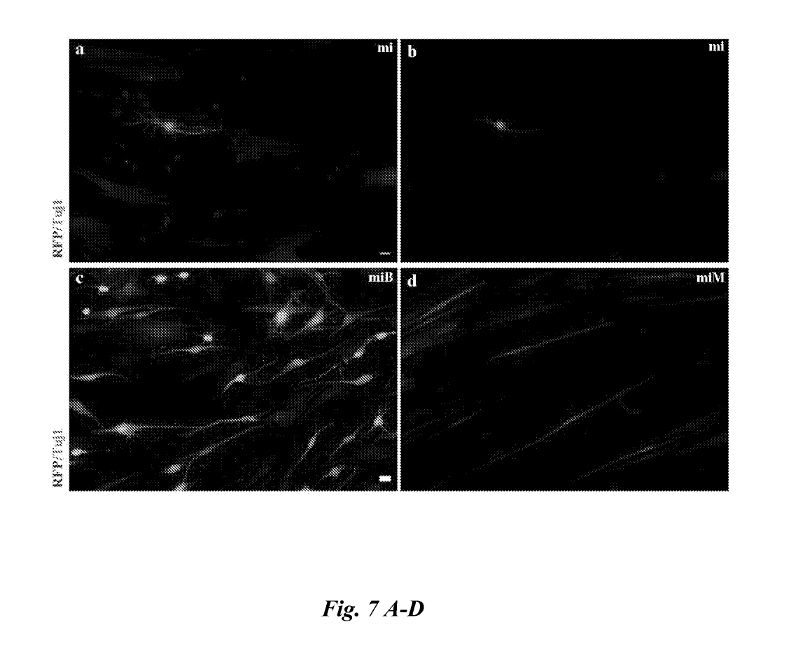

BJ cells were transduced with the individual factors among to 12F pool to determine whether any single factor was sufficient to induce neurons. On day 18 post-transduction, none of the cultures infected with a single factor displayed cells with the distinctive neuronal morphology. In the cells transduced with miR-124 alone we observed a few Tuj1+ cells with cell bodies having small, multiple processes that resembled neurites (FIG. 7a, b). However, even after continued culturing for over a month these cells did not mature further to produce the characteristic neuronal phenotype. Such morphological changes resembled those observed by Yu et al. (Experimental Cell Research (2008) 314:2618-2633) when they overexpressed miR-124 in P19 teratocarcinoma cells. That study also found that treatment with miR-124 plus MASH1 (also known as ASCL1) could induce neurite outgrowth in P19 cells. An additional study reported that introduction of miR-124 into HeLa cells could transform their gene expression pattern to resemble that of brain tissue. Lim, L. P. et al., Nature (2005) 433:769-773. Therefore, miR-124 was combined with single factors in an attempt to recapitulate our observations with the complete 12F pool. Two-factor pools were generated, each composed of miR-124 plus another factor from the 11 transcription factor pool.

No neuronal-like cells were detectable under these methods with any of the two-factor combinations. However, in three of these combinations—miR-124 plus BRN2 (designated miB), miR-124 plus MYT1L (miM), and miR-124 plus PAX 6 (miP)—neuronal-like morphology was observed in many RFP/Tuj1 double-positive cells when compared to controls (FIG. 7c, d). Specifically, miB- and miP-treated cells exhibited a morphology similar to that observed with miR-124 alone, but there was a >20 fold increase in the number of such cells (FIG. 7c). miM treated cells showed a distinctive elongated morphology, unique amongst the combinations that we used (FIG. 7d).

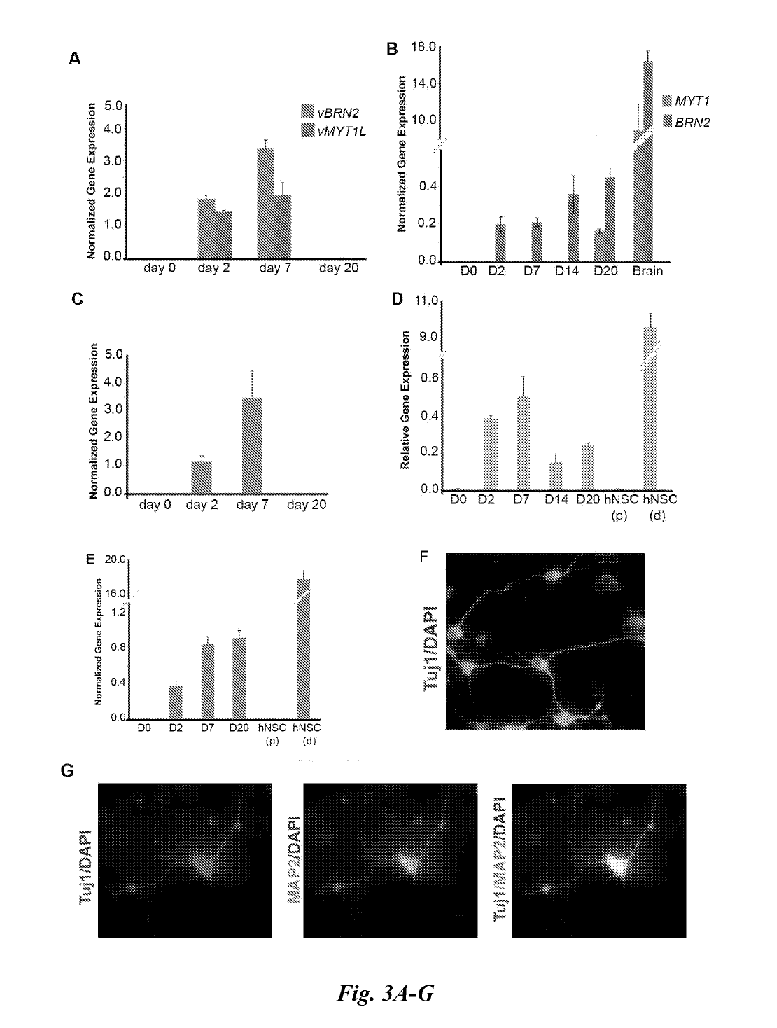

Further three factor combinations were tested from these four factors. Three days after transduction with lentiviruses carrying the miBM transgenes (3F), many RFP-positive BJ cells exhibited small and compact cell bodies with mono- or bipolar projections and weak βIII-tubulin expression (FIG. 1c). A characteristic neuronal morphology, consisting of multiple neuritic extension and elaborate branching, was observed when these cells were allowed to mature for an additional 15 days (FIG. 1d-f). When stained on day 18, these cells displayed positive immunoreactivity for the mature neuronal markers MAP2 (55%, n=100) (FIG. 1g) and NeuN (46%, n=100) (FIG. 1h). In contrast, characteristic mature neuronal cells were not observed under the miPM or miPB conditions, although the resulting cells morphologically resembled those of miP- or miB-transduced cells. Additionally, characteristic mature neuronal cells were not observed in control cultures where miR-124 was replaced with scrambled non-specific small RNAs (FIG. 1i). Similar results were obtained in CRL2097 cells as compared to BJ cells. Clear morphological changes in miBM cultures were observed within ˜3 days of initial viral infection; thus, continued expression of the transgenes may not be essential, in some circumstances, to produce the hiN phenotype in human fibroblasts. After <7 days of combined expression of BRN2, MYT1L and miR-124 using a doxycycline-inducible or cumate-inducible system, hiN cells were produced at a frequency comparable to experiments using non-inducible expression systems (FIG. 1a and FIG. 3a-g). Because the miBM combination robustly generated characteristic neurons for all differentiated non-neuronal cells tested, hiN cells induced under these conditions were characterized in more detail.

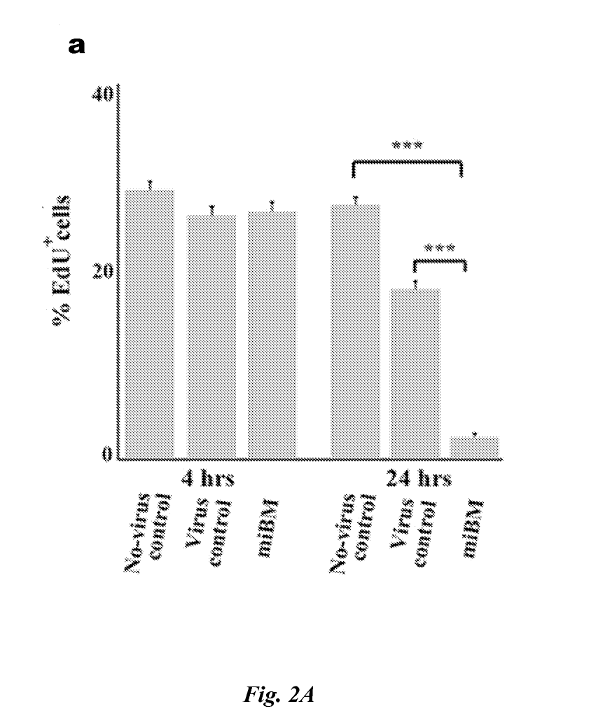

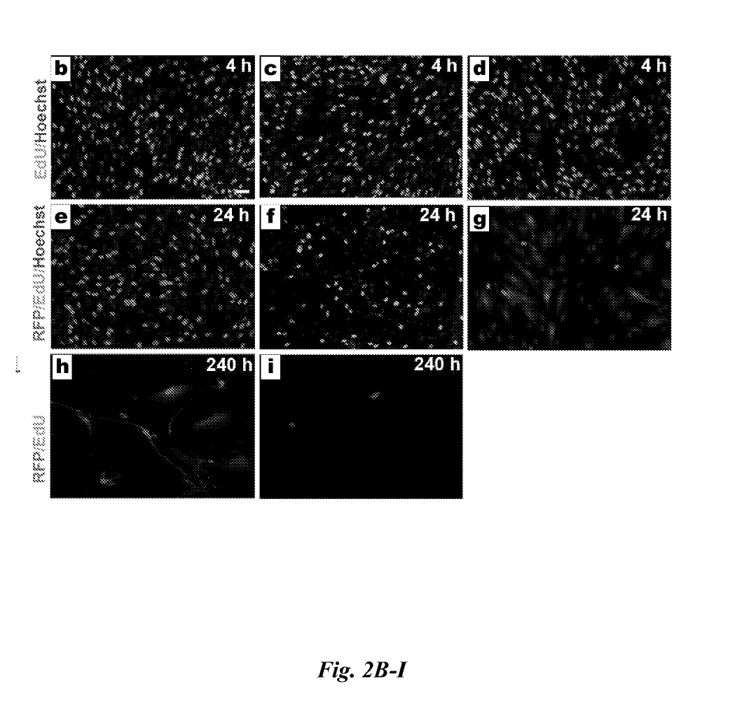

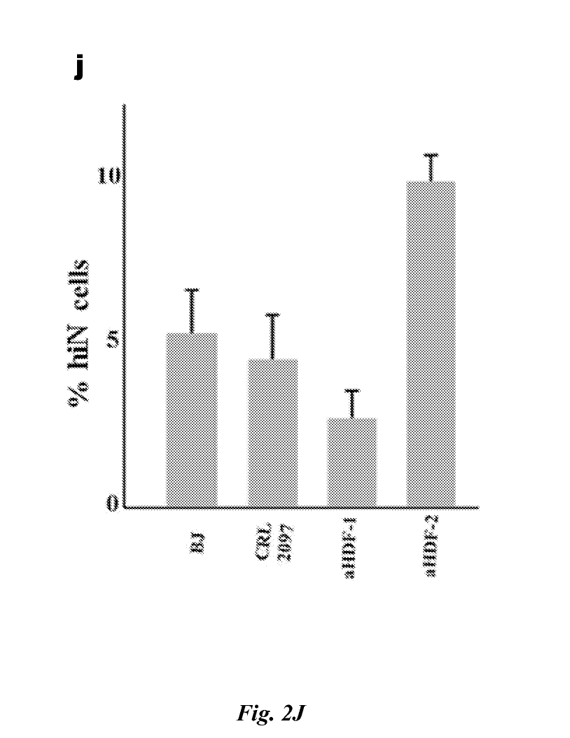

The efficiency of conversion of human fibroblasts to hiN cells was estimated using an EdU incorporation assay. Cells were cultured in the presence of EdU to assess cell division during the conversion process. Cultures pulsed with EdU for two hours and stained at 4 hours post-infection were over 25% EdU positive in both uninfected control and miBM-treated cultures (FIG. 2a-d). In contrast, when the cultures were examined at 24 hours post-infection, immediately after a 2 hour pulse of EdU, miBM cultures manifested <1% dividing cells, while control cultures exhibited >25% EdU-positive cells (FIG. 2a, e-g). In cultures that were maintained in the presence of EdU for 10 days, the vast majority of RFP⁺ cells exhibiting neuronal morphology were negative for EdU staining (FIG. 2h, i). These results suggested that fibroblasts destined to become hiN cells were most likely postmitotic within 24 hours of transgene induction. These data support the finding that miBM induces direct conversion of fibroblasts to neurons without apparent involvement of a mitotic progenitor cell stage, i.e., without dedifferentiation. An efficiency of 4-8% for hiN generation from BJ or CRL2097 human fibroblast cells was estimated by dividing the total number of Tuj1-positive hiN cells on day 18 by the total number of cells in the starting fibroblast population (FIG. 2j).

### hiN Cells Displayed Neurotransmitter Responses and Synaptic Properties Similar to Functional Neurons

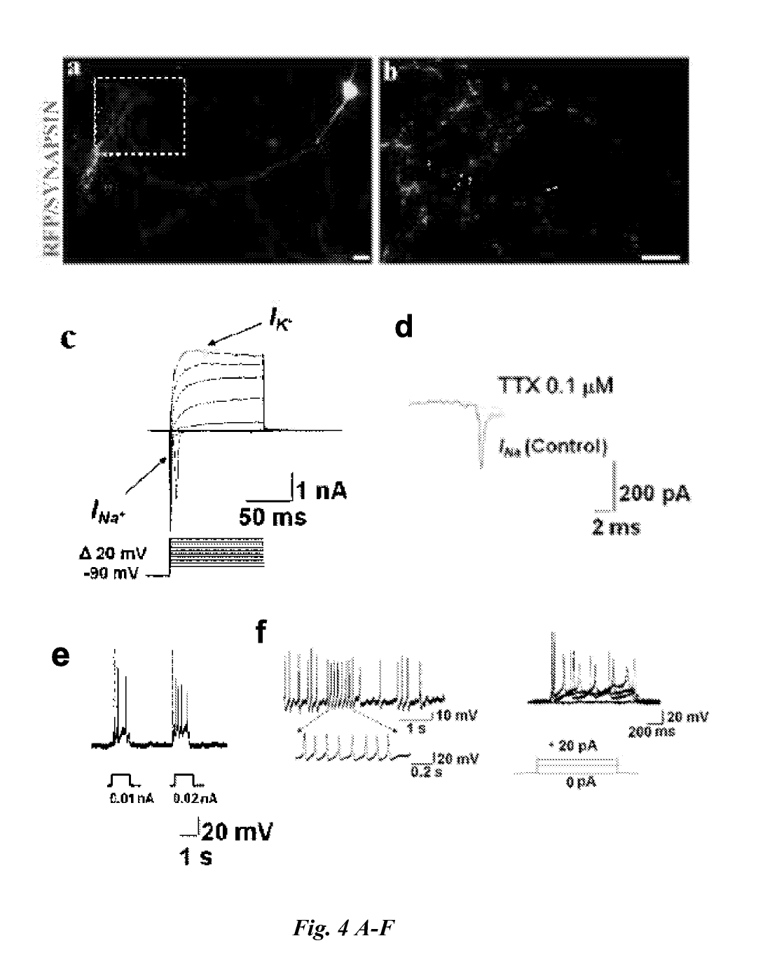

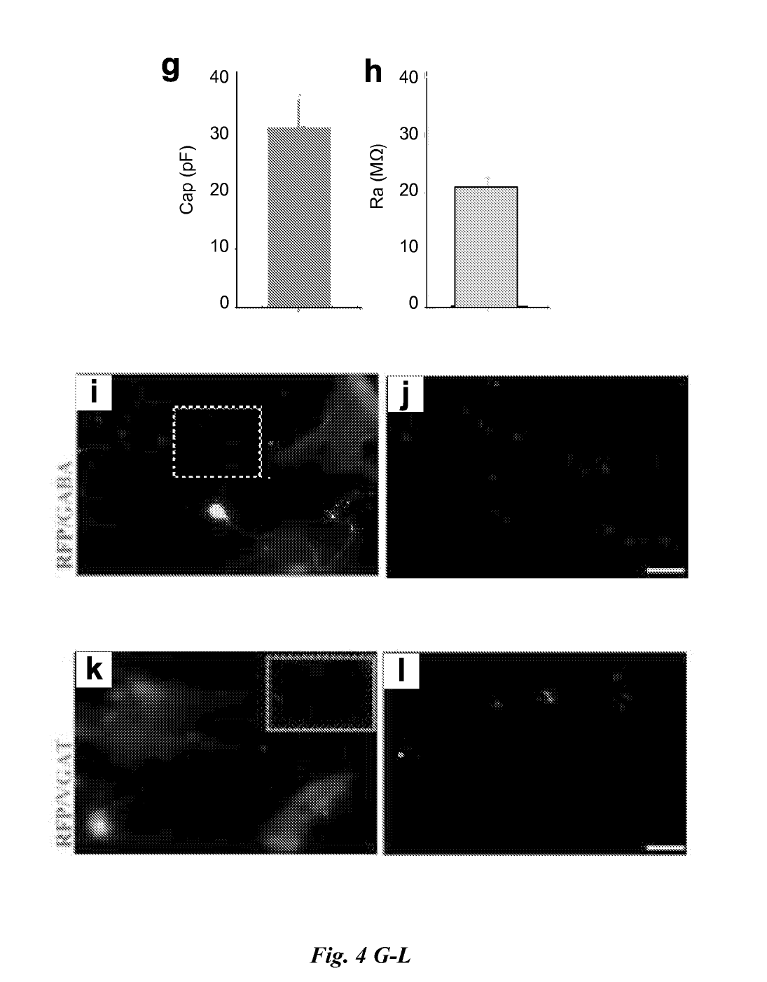

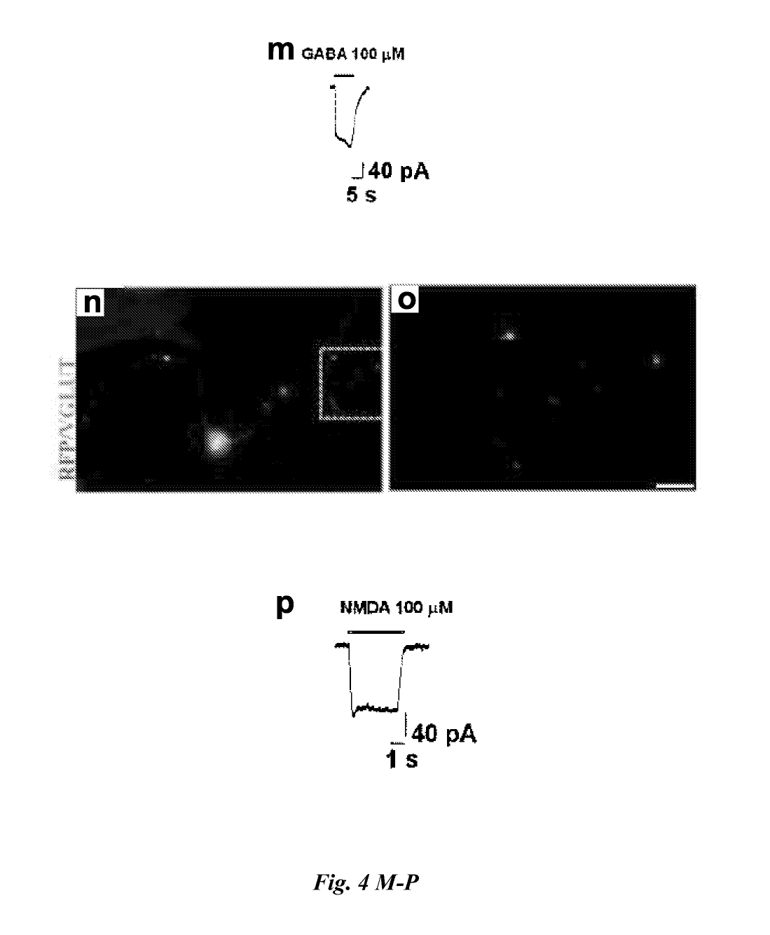

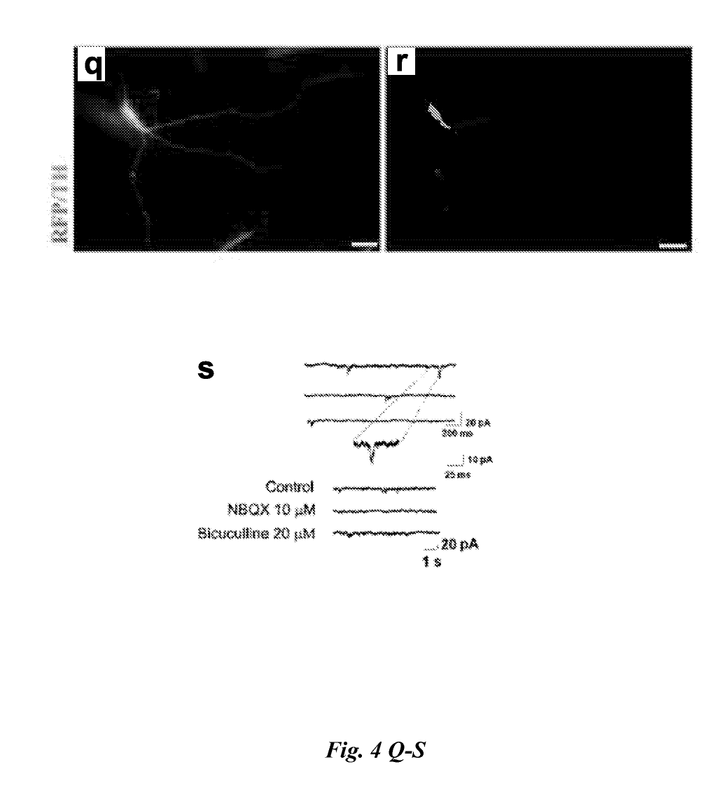

hiN cells derived under miBM condition were functionally characterized by electrophysiology. Patch-clamp recordings on RFP⁺ hiN cells 25 days post-infection were used to assess the membrane properties of hiN cells. At this stage, the cells displayed immunoreactivity for synapsin (FIG. 4a, b), a marker associated with functional maturation of neuronal synapses. During whole-cell recording in the voltage-clamp mode, the majority of hiN cells (60%, n=10) exhibited rapidly inactivating inward current with a rise time of 2-3 ms, followed by outward currents, properties consistent with the opening of voltage dependent Na⁺ and K⁺ channels, respectively (FIG. 4c). The inward current was inhibited by the sodium channel blocker tetrodotoxin (FIG. 4d). In the current-clamp mode, the resting membrane potentials ranged between −37 mV and −42 mV, and the majority of cells (81%, n=29) fired action potentials (amplitude range: −32 mV to −61 mV) during injection of 10 to 20 pA currents (FIG. 4e). Approximately 15% of the recorded cells exhibited spontaneous action potentials (FIG. 4f, left panel), and approximately 20% of cells exhibited repetitive trains of evoked action potentials (FIG. 4f, right panel). Additional electrophysiological parameters, including membrane capacitance (FIG. 4g) and membrane access resistance (FIG. 4h), indicated the functional maturation of the hiN cells.

Functional neurotransmitter properties of hiN cells were examined by testing for specific markers and corresponding ligand-gated currents. Immunostaining revealed that numerous hiN cells were positive for the inhibitory neurotransmitter GABA (FIG. 4i, j), in addition to punctuate staining for VGAT (FIG. 4k, l), a protein involved in vesicular transport of GABA. hiN cells responded to exogenous application of GABA, producing whole-cell currents (FIG. 4m). Additionally, hiN cells displayed properties of excitatory glutamatergic neurons, as indicated by positive VGLUT1 staining (FIG. 4n, o) and slowly decaying NMDA receptor-mediated current in 60% (n=5) of the cells recorded (FIG. 4p). The absence of peripheral neuronal features was suggested by negative immunoreactivity for Peripherin. Occasional cells that were tyrosine hydroxylase-positive were also observed, suggesting a possible dopaminergic-like phenotype (FIG. 4q, r), but none of the cells stained for choline acetyltransferase or serotonin.

The functional synaptic properties of hiN cells were also examined. Patch-clamp recordings were made from hiN cells cultured for 30 days as well as from cells that were trypsinized on day 10 post-infection and then seeded onto a monolayer of human primary astrocytes. Miniature excitatory postsynaptic currents (mEPSCs) were observed, indicative of functional synapses, in 25% of cells (n=8) recorded at a holding potential of −80 mV (FIG. 4s, top panel). These currents were sensitive to NBQX, an AMPA-type glutamate receptor antagonist, but not bicuculline, a competitive inhibitor of GABA_{A} receptors (FIG. 4s, bottom panel), thus further confirming their excitatory nature. The above results collectively provide strong evidence that hiN cells made by the inventive methods herein were functional neurons.

Adult Human Fibroblasts were Directly Reprogrammed to hiN Cells

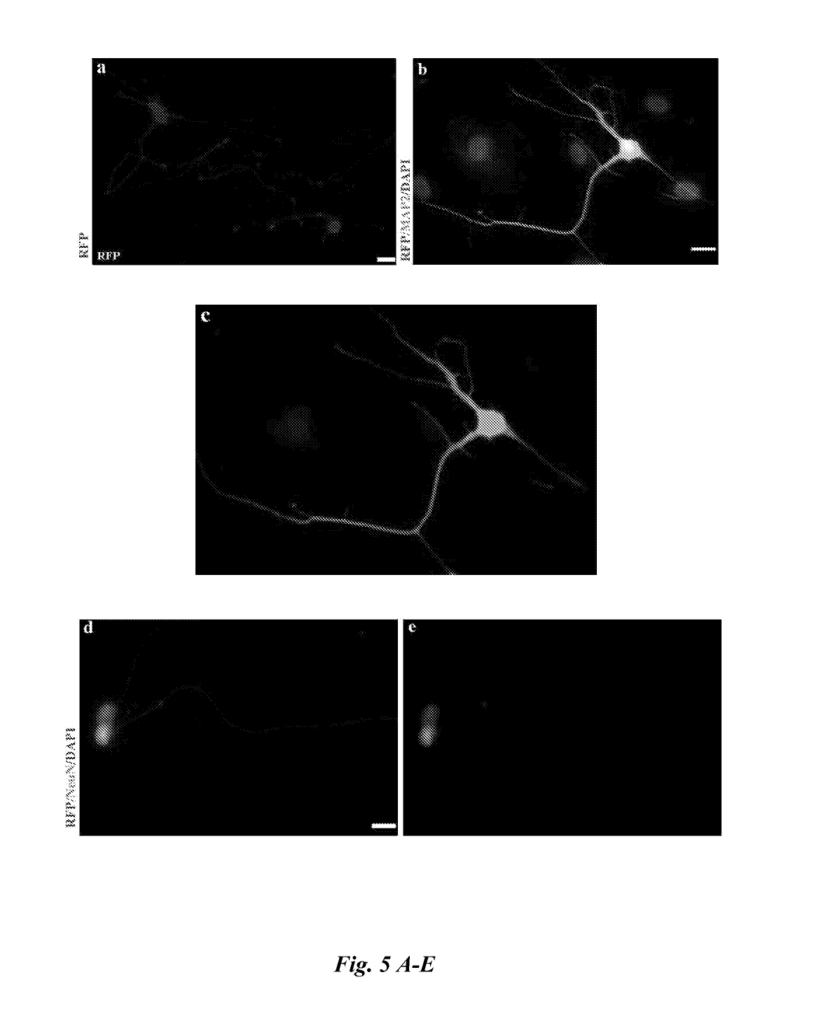

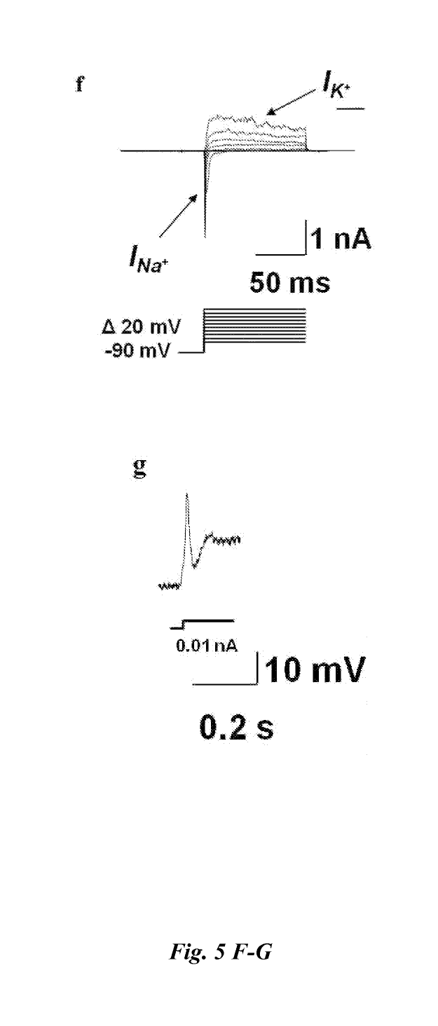

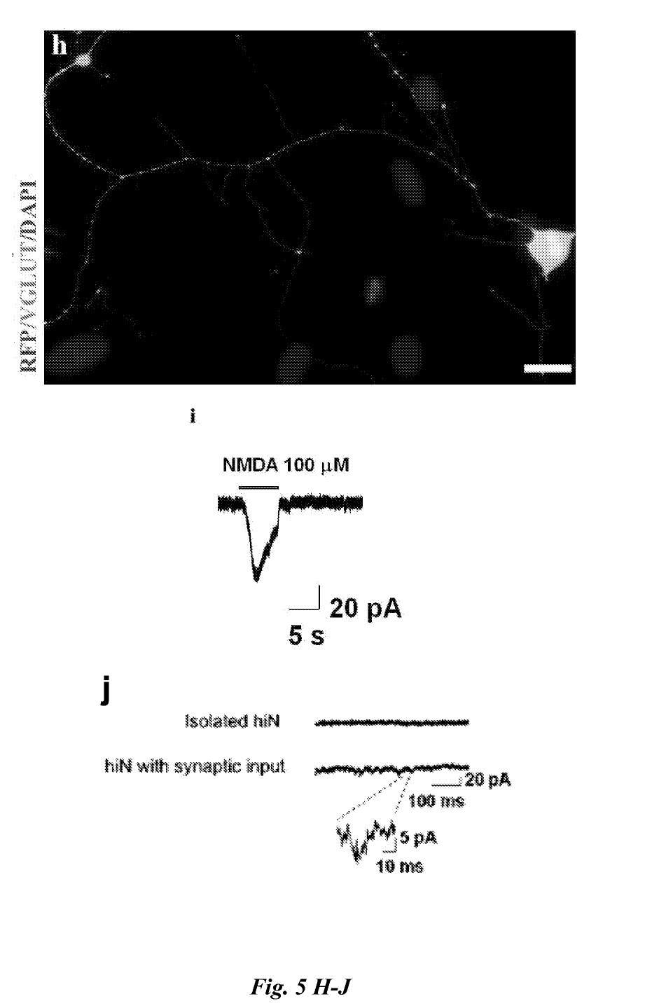

Adult human dermal fibroblasts (aHDFs) derived from a 55 year-old Caucasian female were directly reprogrammed to hiN cells. Similar to BJ and CRL2097 cells, aHDF cells were confirmed free of contaminating neural progenitor cells or neurons (FIG. 6a, b). aHDF cells were transduced with viruses carrying miBM transgenes. aHDF cells were converted to hiN cells with an efficiency of 1.5-2.9% (FIG. 5a), and displayed characteristic mature neuronal morphology and marker genes expression (FIG. 5b-e). aHDF cells also exhibited rapidly inactivating Na⁺ currents (47%, n=17) (FIG. 5f), and could fire action potentials (12%, n=25) (FIG. 5g) when tested on day 25 post-infection. Values for resting membrane potential, membrane capacitance, access resistance, and total membrane resistance were generally comparable to those of BJ or CRL2097-derived hiN cells (Table 2). Moreover, these cells stained positive for VGLUT1 (28%, n=50) (FIG. 5h) and manifested whole-cell currents in response to NMDA (60%, n=5) (FIG. 5i); and thus displayed excitatory neuronal properties. Additionally, when plated at high density, but not at low density, aHDF-hiN cells displayed excitatory synaptic currents (43%, n=7) (FIG. 5j), reflecting functional contacts with neighboring hiNs.

TABLE 2
Electrophysiological membrane properties of hiN cells

| | | | |
|---|---|---|---|
| Property | n | Mean | SEM |

Membrane properties of neonatal fibroblast derived hiN cells

| | | | |
|---|---|---|---|
| Capacitance | 21 | 30.50034 | ±5.46654 |
| Membrane resistance | 21 | 405.97586 | ±56.68332 |
| Access resistance | 21 | 20.07241 | ±1.68852 |

Membrane properties of adult fibroblast derived hiN cells

| | | | |
|---|---|---|---|
| Capacitance | 20 | 35.9135 | ±11.52301 |
| Membrane resistance | 20 | 1067.315 | ±347.43712 |
| Access resistance | 20 | 19.56 | ±1.39498 |

### Methods

Molecular Cloning, Cell Culture and hiN Cell Generation.

Complementary DNAs of the transcription factors were purchased from Open Biosystems or Invitrogen and subcloned into pLenti V5-dest (Invitrogen). Doxycycline inducible plasmids pBrn2-TetO-FUW and pMyt1l-TetO-FUW were a kind gift of Marius Wernig (Stanford University). The pLemir-miR-124 vector was purchased from Open Biosystems. Viral packaging was performed in 293T cells, and fibroblasts cells were infected as previously described (Lin, T. et al., A chemical platform for improved induction of human iPSCs, Nature Methods (2009) 6:805-808).

Cell Culture and hiN Cell Generation.

Human primary fibroblast cells, including BJ, CRL2097 (foreskin dermal fibroblast; both from ATCC) aHDF-1 (ScienCell), and aHDF-2 (PromoCell), were cultured in DMEM containing 10% FBS, MEM non-essential amino acids, Glutamax and 5 mM HEPES. For all the experiments, 1.5×10⁴ (24 well plate) or 5×10⁴ (6 well plate) cells of early passage number (P2-P5) were split and cultured overnight on poly-lysine (Sigma) or poly-ornithine plus poly-laminin (Sigma) coated dishes prior to infection with lentiviral particles. The infected cells were then cultured in fibroblast medium for 24 hours before changing to N4 medium (induction medium) (DMEM:F12, N2 supplement, B27 supplement, 5 mM HEPES, 0.5% Albumax, 0.6% glucose and MEM non-essential amino acids (all from Invitrogen), plus 20 ng ml⁻¹ bFGF (R&D Systems) and 100 ng ml⁻¹ human-recombinant Noggin (Stemgent)). Doxycycline (2 Ξml⁻, Sigma) was added for 4-7 days at each media change (every other day). Subsequently, cells were cultured in neuronal maturation medium (neuronal differentiation medium) (DMEM:F12, N2 supplement, B27 supplement, 5 mM HEPES, 0.5% Albumax, 0.6% glucose and non-essential amino acids (all from invitrogen), plus 20 ng ml⁻¹ GDNF (R&D Systems), 10 ng ml⁻¹ BDNF (R&D Systems) and 3 mg ml⁻¹ Forskolin (Tocris)) until they were used for electrophysiology experiments or fixed for immunostaining.

For calculating the efficacy of hiN cell conversion from fibroblasts, the following technique was used. The number of hiN cells was calculated by scoring 20 randomly-selected visual fields under a 20× objective. The total surface area of the field was then measured, allowing us to estimate the density of neurons per field and thus estimate the total number of neurons in the entire well. This number was then divided by the total number of cells seeded in the well to obtain an estimate of the percentage of conversion.

Antibodies, Immunostaining and EdU Labeling.

Primary antibodies used included: anti-Tuj1 (Covance, 1:1000), chicken anti-MAP2 (Abcam, 1:5000), mouse anti-NeuN (wMillipore, 1:100), rabbit anti-Synapsin1 (Millipore, 1:2000), rabbit anti-GABA (Sigma, 1:1000), guinea pig anti-VGLUT1 (Synaptic Systems, 1:2000), mouse anti-VGAT (Synaptic Systems, 1:500), mouse anti-TH (Sigma, 1:1000), and mouse anti-peripherin (Millipore, 1:50). Alexa-350-, Alexa-488- and Alexa-555-conjugated secondary antibodies were purchased from Invitrogen. Immunostaining was performed as previously described (Lin, T. et al., A chemical platform for improved induction of human iPSCs, Nature Methods (2009) 6:805-808). For the experiments assessing cell proliferation, 10 μM EdU was added to the culture medium and were either pulsed for two hours, or maintained throughout the duration of the culture (up to 10 days). EdU staining was performed as per the manufacturer's instructions (Invitrogen), and cells were counterstained with Hoechst 33258 to identify nuclei.

RT-PCR.

RNA was isolated using RNeasy minikit (Qiagen), and 1 μg of each sample was reverse transcribed using iScript (Biorad). Two microliter aliquots of the resulting cDNA were used for PCR with Platinum Taq DNA polymerase (invitrogen). The sequences of the primers used for PCR were as follows: SOX1 forward:

5′ACTCCAGGGTACACACAGGG3′ (SEQ ID NO:8); SOX1 reverse:

5′TCTGTTAACTCACCGGGACC3′ (SEQ ID NO:9); SOX 10 forward:

5′CGCTTGTCACTTTCGTTCAG3′ (SEQ ID NO:10); SOX 10 reverse:

### 5′CCTTCATGGTGTGGGCTC3′ (SEQ ID NO:11).

Electrophysiology.

Twenty-four hours after viral transduction, infected cells were trypsinized and plated on poly-ornithine and poly-laminin coated glass coverslips (12 mm) and then further cultured in neuronal maturation medium. Coverslips were placed in the recording chamber mounted on an Olympus IX 71 microscope. Spontaneous or evoked responses were recorded at room temperature (22±1° C.) via whole-cell recording with a patch electrode. Signals were amplified using an Axopatch200B (Axon Instruments) and filtered with 2 KHz via Bessel low-pass filter. Data were sampled and analyzed using pClamp10.1 software in conjunction with a DigiData interface (Model 1322A, Axon Instruments). Patch pipettes were pulled from the standard wall glass of 1.5 mm OD (Warner Instruments, USA) and had input resistances of 5-11 MΩ.

For recording voltage-gated currents and action potentials, patch electrodes were filled with the following solution (in mM): 140 K-gluconate, 5 NaCl, 1 MgCl₂, 10 EGTA, 10 HEPES, 10 EGTA, pH adjusted by KOH to 7.25, osmolarity measured at 290 mOsm. The composition of the intracellular solution used for recording ligand-gated currents was as follows (in mM): 130 Cs-gluconate; 2 MgATP, 1 MgCl₂; 10 EGTA; 10 HEPES, pH 7.25, osmolarity 300 mOsm. The bath solution generally contained a Na ‘saline based upon Hanks’ balanced salt solution (pH 7.3).

To monitor voltage-gated currents, after an initial pre-hyperpolarization to −90 mV for 300 ms to relieve inactivation, step potentials of ±20 mV, ranging from −60 to +30 mV, for 100 ms were applied. Drugs were prepared in bath solution and applied by an array of microtubes placed 75-100 μm from the cells. Solution changes were achieved rapidly, within 50-100 ms, by moving the array of constantly flowing pipette tips relative to the cell with a micromanipulator driver. A control pipette containing bath solution was used to rapidly wash off applied drugs. N-methyl-D-aspartate (NMDA) and tetrodotoxin (TTX)) were purchased from Tocris; γ-aminobutyric acid (GABA) was purchased from Sigma. For recording synaptic activity, mESPCs were monitored under voltage-clamp in the presence of 1 μM TTX at a holding potential of −80 mV.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of generating a neuronal cell from a differentiated non-neuronal cell, the method comprising:
increasing the amount of a miR-124 microRNA, a MYT1L transcription factor, and a BRN2 transcription factor in the differentiated non-neuronal cell; and
culturing the differentiated non-neuronal cell in conditions suitable for neuronal differentiation; thereby generating the neuronal cell from the differentiated non-neuronal cell.

2. The method of claim 1, wherein the differentiated non-neuronal cell is
(a) a human cell;
(b) a fibroblast cell;
(c) an adult cell; or
(d) a neonatal cell.

3. (canceled)

4. The method of claim 2, wherein the differentiated non-neuronal cell is a dermal fibroblast cell.

5-6. (canceled)

7. The method of claim 1, wherein:
(a) the miR-124 microRNA comprises an oligonucleotide sequence that is at least 95% identical to any of SEQ ID NOs:1 or 4-6;
(b) the MYT1L transcription factor comprises an amino acid sequence that is at least 90% identical to SEQ ID NO:2; or
(c) the BRN2 transcription factor comprises an amino acid sequence that is at least 90% identical to SEQ ID NO:3.

8. The method of claim 7, wherein:
(a) the miR-124 microRNA comprises SEQ ID NO:1, SEQ ID NO:4, SEQ ID NO:5, or SEQ ID NO:6;
(b) the MYT1L transcription factor comprises SEQ ID NO:2; or
(c) the BRN2 transcription factor comprises SEQ ID NO:3.

9-12. (canceled)

13. The method of claim 1, wherein the amount of one or more of the miR-124 microRNA, MYT1L transcription factor, and BRN2 transcription factor is increased by introducing into the differentiated non-neuronal cell one or more of a first, second, and third expression cassette,
the first expression cassette comprising a promoter operably linked to a polynucleotide encoding the miR-124 microRNA;
the second expression cassette comprising a promoter operably linked to a polynucleotide encoding the MYT1L transcription factor; and
the third expression cassette comprising a promoter operably linked to a polynucleotide encoding the BRN2 transcription factor.

14. The method of claim 13, wherein:
(a) two or more of the first, second, and third expression cassettes are introduced into the differentiated non-neuronal cell;
(b) each of the first, second, and third expression cassettes is introduced into the differentiated non-neuronal cell;
(c) the promoters of the first, second, and third expression cassettes are different;
(d) the promoters of at least two of the first, second, and third expression cassettes are the same promoter;
(e) the promoter is an inducible promoter;
(f) the expression cassette is introduced to the cell as part of a viral vector;
(g) one or more of the polynucleotide encoding miR-124, the polynucleotide encoding MYT1L, and the polynucleotide encoding BRN2 is transiently expressed in the differentiated non-neuronal cell; or
(h) one or more of the polynucleotide encoding miR-124, the polynucleotide encoding MYT1L, and the polynucleotide encoding BRN2 is stably expressed in the differentiated non-neuronal cell.

15-19. (canceled)

20. The method of claim 13, wherein the viral vector is a lentiviral vector or an adenoviral vector.

21-22. (canceled)

23. The method of claim 1, wherein the amount of the miR-124 microRNA is increased by introducing into the differentiated non-neuronal cell a polynucleotide encoding the miR-124 microRNA.

24. The method of claim 1, wherein the amount of one or more of the MYT1L transcription factor and BRN2 transcription factor is increased by introducing into the differentiated non-neuronal cell one or more of a MYT1L polypeptide and a BRN2 polypeptide.

25. The method of claim 1, wherein the neuronal cell is a neuron, an excitatory neuron, an excitatory neuron that produces an N-Methyl-D-aspartic acid (NMDA) current, an inhibitory neuron, a functional neuron, or a mature neuron.

26-28. (canceled)

29. The method of claim 1, wherein:
(a) the amount of at least one of miR-124 microRNA, MYT1L transcription factor, or BRN2 transcription factor in the differentiated non-neural cell is increased for no more than 7 days; or
(b) the amount of at least one of miR-124 microRNA, MYT1L transcription factor, or BRN2 transcription factor in the differentiated non-neuronal cell is increased for no more than 4 days.

30. (canceled)

31. The method of claim 1, wherein:
(a) the conditions that induce neuronal differentiation are chemically defined conditions; or
(b) the culturing step comprises contacting the differentiated non-neuronal cell with at least one of bFGF or Noggin.

32. (canceled)

33. The method of claim 31, wherein the culturing step further comprises contacting the differentiated non-neuronal cell with one or more of GDNF, BDNF, and forskolin.

34. (canceled)

35. The method of claim 25, wherein the time from initiating the increase of miR-124, MYT1L, and BRN2 to the generation of the functional neuron is no more than 25 days.

36. (canceled)

37. The method of claim 25, wherein the time from initiating the increase of miR-124, MYT1L, and BRN2 to the generation of the mature neuron is no more than 20 days.

38. The method of claim 25, wherein the time from initiating the increase of miR-124, MYT1L, and BRN2 to the generation of the mature neuron is no more than 18 days.

39. The method of claim 1, wherein following the culturing step:
(a) the method further comprises screening the differentiated non-neuronal cell for the presence of at least one neuronal marker; or
(b) the method further comprises screening the differentiated non-neuronal cell for the production of an electrical current.

40. The method of claim 39, wherein the at least one neuronal marker is MAP2, NeuN, synapsin, GABA, VGAT, or VGLUT1.

41. (canceled)

42. The method of claim 1, wherein the method is conducted at least partly in vivo.

43. The method of claim 1, wherein the method is conducted in vitro.

44. A neuronal cell generated by the method of claim 1.
